SEQUENCE LISTING (7 sequences)

SEQ ID NO: 1 - DNA, 1467 bp, Homo sapiens
atggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggacgcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccgctactgatgcaggcactgcaggagccctgactccacagcatgttcgagctcatgccgctccagctgctctgcagttgggagctgtttctcctgggacactgacccccactggagtagtctctggcccagcagctacacccacagctcagcatcttcgacaggctgcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcaggtcctcttcctgatggatgggaacaagccatgactcaggatggagaaatttactatataaaccataagaacaagaccacctcttggctagacccaaggcttgaccctcgttttgccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgaggctacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatag

SEQ ID NO: 2 - DNA, 1467 bp, Homo sapiens
atggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggactcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccagtactgatgcaggcactgcaggagccctgactccacagcatgttcgagctcatgcctctccagcttctctgcagttgggagctgtttctcctgggacactgacccccactggagtagtctctggcccagcagctacacccacagctcagcatcttcgacagtcttcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcaggtcctcttcctgatggatgggaacaagccatgactcaggatggagaaatttactatataaaccataagaacaagaccacctcttggctagacccaaggcttgaccctcgttttgccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgagagtacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatag

SEQ ID NO: 3 - DNA, 1467 bp, Homo sapiens
atggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggactcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccagtactgatgcaggcactgcaggagccctgactccacagcatgttcgagctcattcctctccagcttctctgcagttgggagctgtttctcctgggacactgacccccactggagtagtctctggcccagcagctacacccacagctcagcatcttcgacagtcttcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcaggtcctcttcctgatggatgggaacaagccatgactcaggatggagaaatttactatataaaccataagaacaagaccacctcttggctagacccaaggcttgaccctcgttttgccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgagagtacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatag

SEQ ID NO: 4 - DNA, 1467 bp, Homo sapiens
tacctagggcccgtcgtcggcggcggagttggccggggggtcccggttcccgtcggcggaagcgtcgggggcgtccccgtcccgggcggcaggcctgggcccgttggccgtgggcgccgctgggtccgccgcggcgtccgtggggggcggcccgtagtctagcacgtgcaggcgcccctgcgcctctggctggacctccgcgagaagttgcggcagtacttggggttctgccggttgcacggggtctggcacgggtactccgaggccttcgacgggctgaggaagaagttcggcggcctcgggtttagggtgagggctgtccggcgatgactacgtccgtgacgtcctcgggactgaggtgtcgtacaagctcgagtacggcgaggtcgacgagacgtcaaccctcgacaaagaggaccctgtgactgggggtgacctcatcagagaccgggtcgtcgatgtgggtgtcgagtcgtagaagctgtccgacgaaaactctatggactactacatggagacggtcgtccaaccctctaccgtttctgtagaagaccagtctctatgaagaatttagtgtagctagtctgttgttgtaccgtcctggggtccttccggtacgacagggtctacttgcagtgtcgggggtggtcaggtggtcacgtcgtcttatactacttgagccgaagtccaggagaaggactacctacccttgttcggtactgagtcctacctctttaaatgatatatttggtattcttgttctggtggagaaccgatctgggttccgaactgggagcaaaacggtacttggtctcttagtcagtctcacgaggtcactttgtcggtggtggggaccgaggggtctcgggtgtccctccgcagtacccaccgtcgttgaggttggtcgtcgttgtctacgctgacgtcgttgacgtctacctcttcctctccgacgccgactttgtcgttcttgacgaagccgtcctcaatcgggacgcatcggtcaatggttgtgacctcgtcctaccaccctgagttttaggtcacagaagagggccctacagagtccttaactcttgttactgctggttatcgagtctaggaaaggaattgtcaccgtggatagtgagagctctactccgatgtctgtcacctgattcgtactcgtcgatgtcacagggagcttggggtctactgaaggacttgtcacacctactctacctatgtccactatgatagttggtttcgtgggacgggagtgtcgtcttggcaaagggtctgatggaacttcggtaaggaccctgtttacacctggaaccttgtgaccttcctctaccttacttgtatcttcctctcctcgactacggttcagacgtccttcgaaactcaagactgtaggaattactgtacctcagacaaaaccgacggtggttcgatctatttctttcgaaagaatgtaccaatatc

SEQ ID NO: 5 - DNA, 1467 bp, Homo sapiens
tacctagggcccgtcgtcggcggcggagttggccggggggtcccggttcccgtcggcggaagcgtcgggggcgtccccgtcccgggcggcaggcctgggcccgttggccgtgggcgccgctgggtccgccgcggcgtccgtggggggcggcccgtagtctagcacgtgcaggcgcccctgagcctctggctggacctccgcgagaagttgcggcagtacttggggttctgccggttgcacggggtctggcacgggtactccgaggccttcgacgggctgaggaagaagttcggcggcctcgggtttagggtgagggctgtccggtcatgactacgtccgtgacgtcctcgggactgaggtgtcgtacaagctcgagtacggagaggtcgaagagacgtcaaccctcgacaaagaggaccctgtgactgggggtgacctcatcagagaccgggtcgtcgatgtgggtgtcgagtcgtagaagctgtcagaagaaaactctatggactactacatggagacggtcgtccaaccctctaccgtttctgtagaagaccagtctctatgaagaatttagtgtagctagtctgttgttgtaccgtcctggggtccttccggtacgacagggtctacttgcagtgtcgggggtggtcaggtggtcacgtcgtcttatactacttgagccgaagtccaggagaaggactacctacccttgttcggtactgagtcctacctctttaaatgatatatttggtattcttgttctggtggagaaccgatctgggttccgaactgggagcaaaacggtacttggtctcttagtcagtctcacgaggtcactttgtcggtggtggggaccgaggggtctcgggtgtccctccgcagtacccaccgtcgttgaggttggtcgtcgttgtctacgctgacgtcgttgacgtctacctcttcctctccgacgccgactttgtcgttcttgacgaagccgtcctcaatcgggacgcatcggtcaatggttgtgacctcgtcctaccaccctgagttttaggtcacagaagagggccctacagagtccttaactcttgttactgctggttatcgagtctaggaaaggaattgtcaccgtggatagtgagagctctactctcatgtctgtcacctgattcgtactcgtcgatgtcacagggagcttggggtctactgaaggacttgtcacacctactctacctatgtccactatgatagttggtttcgtgggacgggagtgtcgtcttggcaaagggtctgatggaacttcggtaaggaccctgtttacacctggaaccttgtgaccttcctctaccttacttgtatcttcctctcctcgactacggttcagacgtccttcgaaactcaagactgtaggaattactgtacctcagacaaaaccgacggtggttcgatctatttctttcgaaagaatgtaccaatatc

SEQ ID NO: 6 - protein, 488 aa, Homo sapiens
MDPGQQPPPQPAPQGQGQPPSQPPQGQGPPSGPGQPAPAATQAAPQAPPAGHQIVHVRGDAETDLEALFNAVMNPKTANVPQTVPMRLRKLPDSFFKPPEPKSHSRQAATDAGTAGALTPQHVRAHAAPAALQLGAVSPGTLTPTGVVSGPAATPTAQHLRQAAFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQELALRSQLPTLEQDGGTQNPVSSPGMSQELRTMTTNSSDPFLNSGTYHSRDEATDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 7 - protein, 488 aa, Homo sapiens
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHASPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

(19) United States
(12) Patent Application Publication
(10) Pub. No.: US 2021/0324412 A1
(43) Pub. Date: Oct. 21, 2021

(54) ACTIVATION OF YAP SIGNALING FOR SENSORY RECEPTOR REGENERATION

(71) Applicant: UNIVERSITY OF SOUTHERN CALIFORNIA, Los Angeles, CA (US)

(72) Inventors: Ksenia GNEDEVA, Malibu, CA (US); Neil I. SEGIL, Altadena, CA (US); Xizi WANG, Arcadia, CA (US)

(21) Appl. No.: 17/231,328

(22) Filed: Apr. 15, 2021

Related U.S. Application Data
(60) Provisional application No. 63/010,427, filed on Apr. 15, 2020.

(51) Int. Cl. C12N 15/86; A61K 9/00; C12N 7/00; A61P 27/16

(52) U.S. Cl. CPC C12N 15/86; A61K 9/0046; C12N 2750/14171; A61P 27/16; C12N 2750/14143; C12N 7/00

(57) ABSTRACT

A method for inducing sensory receptor regeneration includes a step of identifying a subject in need of regeneration of inner ear sensory epithelia. Yap/Tead signaling in the subject is then activated. Typically, Yap/Tead signaling is activated by introducing an expression vector into the subject such that the expression vector contacts inner ear sensory epithelia in a sufficient amount to induce regeneration thereof. Characteristically, the expression vector encodes a constitutively active YAP gene.

### CROSS-REFERENCE TO RELATED APPLICATIONS

This application claims the benefit of U.S. provisional application Ser. No. 63/010,427 filed Apr. 15, 2020, the disclosure of which is hereby incorporated in its entirety by reference herein.

### STATEMENT REGARDING FEDERALLY SPONSORED RESEARCH OR DEVELOPMENT

The invention was made with Government support under Contract No. 1R21DC016984 awarded by the National Institutes of Health. The Government has certain rights to the invention.

### SEQUENCE LISTING

The text file sequence-usc0282_ST25 of size 18 KB created Apr. 14, 2021 filed herewith, is hereby incorporated by reference.

### TECHNICAL FIELD

In at least one aspect, the present invention is related to methods for regeneration of sensory receptors.

### BACKGROUND

The two major cell types in the sensory organs of the inner ear—hair cells and supporting cells—are derived from the SOX2-positive progenitors specified in the prosensory domain of the otic vesicle (1). In the otolithic vestibular sensory organs, the utricle and the saccule, progenitor cells begin to differentiate into sensory hair cells in the central region of the macula early during embryonic development (2)(3). Concurrent with hair cell differentiation, a wave of cell cycle exit initiates in the macula, spreads towards the periphery of the organ, and gradually restricts progenitor cell proliferation between E11.5 and P2 (2)(3)(4)(5)(6). In contrast to the vestibular sensory epithelia, the auditory organ of Corti undergoes a rapid, 48 hour wave of cell cycle exit that arrests progenitor cell proliferation between E12.5 and E14.5, prior the initiation of differentiation (2)(7)(8).

Despite these differences in the spatiotemporal patterns of cell cycle exit in the vestibular and auditory sensory epithelia, it has been linked to p27^{Kip1} up-regulation in both systems (3)(9)(7). In the organ of Corti, a particularly striking wave of transcriptional activation of CDKN1B gene, coding for p27^{Kip1}, spreads from the apex to the base of the cochlear duct and controls both the timing and the pattern of the cell cycle exit (8). However, what initiates this increase in CDKN1B expression is not understood. In addition, conditional ablation of CDKN1B in the inner ear is not sufficient to completely relieve the block on supporting cell proliferation (9)(10), suggesting the existence of additional repressive mechanisms.

It has previously been demonstrated that the pattern of cell cycle exit and the dynamics of the vestibular sensory organ growth is controlled by a negative feedback mechanism mediated by the Hippo pathway (6). This evolutionarily conserved signaling cascade controls organ growth mainly by repressing cell proliferation (11). Hippo's downstream effector proteins—Yap and Taz—function in a complex with Tead transcription factors to directly activate expression of cell cycle, prosurvival, and antiapoptotic genes (12)(13). Mechanistically, the Yap/Tead complex recruits the Mediator complex to distal regulatory elements of their target genes (14)(15). The molecular output of this signaling is highly tissue- and context-dependent, as evidenced by the large variation observed between Yap/Tead targets in different cancer cell lines, for example (15)(16). However, little is known about the Yap/Tead targetome in developing embryonic tissues in situ, and the role of this transcription factor complex has not been investigated during organ of Corti development.

Although, Yap/Tead signaling is well-known to influence tissue growth and organ size during development, the molecular outputs of the pathway are tissue and context dependent and remain poorly understood.

### SUMMARY

In at least one aspect, the present invention work expands the mechanistic understanding of how Yap/Tead signaling controls the precise number of progenitor cells that will be laid down within the developing inner ear to ultimately regulate the final size and function of the sensory organs.

In another aspect, the first evidence that restoration of hearing and vestibular function may be amendable to YAP-mediated regeneration is provided. The data set forth below shows that re-activation of Yap/Tead signaling after hair cell loss induces proliferative response in vivo—a process thought to be permanently repressed in the mammalian inner ear.

In another aspect, changes in gene expression and chromatin accessibility that occur during cell cycle exit in organ of Corti progenitor cells are characterized. A key role for the Yap/Tead transcription factor complex in maintaining progenitor cell self-renewal and identified many direct target genes of the Yap/Tead complex in this tissue is uncovered. In addition, the results suggest that re-activation of Yap/Tead signaling in the postnatal inner ear sensory epithelia is sufficient to induce a proliferative response, and so can potentially be used as a strategy to promote inner ear sensory organ regeneration.

In still another aspect, a method for inducing sensory receptor regeneration includes a step of identifying a subject in need of regeneration of inner ear sensory epithelia. Yap/Tead signaling in the subject is then activated. Typically, Yap/Tead signaling is activated by introducing an expression vector into the subject such that the expression vector contacts inner ear sensory epithelia in a sufficient amount to induce regeneration thereof. Characteristically, the expression vector encodes a constitutively active YAP gene.

Aspects of the invention show that precise control of organ growth and patterning is executed through a balanced regulation of progenitor self-renewal and differentiation. In the auditory sensory epithelium—the organ of Corti—progenitor cells exit the cell cycle in a coordinated wave between E12.5 and E14.5 prior to initiation of sensory receptor cell differentiation, making it a unique system to study the molecular mechanisms controlling the switch between proliferation and differentiation. The Yap/Tead complex is identified as a key regulator of the self-renewal gene network in organ of Corti progenitor cells. It is also shown that Tead transcription factors bind directly to the putative regulatory elements of many stemness and cell cycle-related genes. The Tead co-activator protein, Yap, is shown to be degraded specifically in the Sox2-positive domain of the cochlear duct, resulting in downregulation of Tead gene targets. Further, conditional loss of the Yap gene in the inner ear results in formation of significantly smaller auditory and vestibular sensory epithelia. The viral gene delivery of Yap5SA, a constitutively active version of Yap, in the postnatal inner ear sensory epithelia in vivo is shown to drive cell cycle re-entry after hair cell loss. Together, these data highlight the key role of Yap/Tead transcription factor complex in maintaining inner ear progenitors during development and suggest new strategies to induce sensory cell regeneration.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a further understanding of the nature, objects, and advantages of the present disclosure, reference should be had to the following detailed description, read in conjunction with the following drawings, wherein like reference numerals denote like elements and wherein:

FIG. 1A. Vector map of the pAAV-CMC-flag-YAP-5SA-EGFP plasmid used in the experiments below.

FIG. 1B. DNA sequence for the YAP5SA gene which is a constitutively active YAP (SEQ ID NO: 1).

FIG. 1C. DNA sequence for the WT YAP gene with 127SA mutation (SEQ ID NO: 2).

FIG. 1D. DNA sequence for the WT YAP gene (SEQ ID NO: 3).

FIGS. 2A, 2B, 2C, 2D, 2E, 2F, and 2G. RNA-sequencing and ATAC-sequencing reveal dramatic changes in the regulation of self-renewal genes in organ of Corti progenitor cells between E12.0 and E13.5. (A) To demonstrate the timing of cell cycle exit in the organ of Corti, an EdU pulse was given 30 min prior to inner ear dissections at E12.0 and E13.5. Immunofluorescence analysis shows that at E12.0 Sox2-positive progenitors incorporate EdU, confirming that these are actively cycling cells (top panels). In contrast, at E13.5 the Sox2-positive progenitor cells upregulate p27Kip1 expression and no longer incorporate EdU (bottom panels). To FACS-purify organ of Corti (OC) progenitor cells before (E12.0) and after (E13.5) the cell cycle exit, Sox2-GFP and p27Kip1-GFP mice were used. Scale bars: 100 m. (B) Principal components analysis of RNA-seq data from E12.0 and E13.5 OC progenitor cells demonstrate that the two replicates collected for each stage cluster tightly with each other. Almost all the variance between E12.0 and E13.5 samples could be explained by the first principal component (PC1=96.25%). (C) Gene ontology (GO) enrichment analysis performed with the DAVID software demonstrates that the term associated with the cell cycle is the most enriched in the genes, differentially expressed (FDR<0.01) between E12.0 and E13.5 in the OC. (D) On the left, the heatmap demonstrates the relative expression levels of 365 cell cycle genes differentially expressed between E12.0 and E13.5 progenitor cells (FDR<0.01; n=2 for each condition). Highly expressed genes are shown in red, while the genes with relatively low levels of expression are depicted in blue. On the right, the bar graphs demonstrate FPKM values of the top up- and down-regulated genes. (E) Heatmap showing differentially accessible chromatin regions determined by ATAC-seq in E12.0 and E13.5 OC progenitor cells was generated using DeepTools. The open chromatin regions, specific to E12.0 (24,530), common between E12.0 and E13.5 (61,498), and specific to the E13.5 (13,352) were identified. (F) Top four transcription factor DNA-binding motifs enriched in the open chromatin regions preferentially accessible at E12.0 and at E13.5 (G) in the OC progenitor cells were identified using Homer motif enrichment analysis. Tead DNA-binding motif is significantly enriched in E12.0-specific regions.

FIGS. 3A, 3B, 3C, 3D, 3E-1, 3E-2, 3E-3, 3E-4, 3F, and 3G. Tead transcription factors directly bound to the putative regulatory elements of many stemness and cell cycle genes. (A) Venn diagram demonstrates the overlaps between E12.0 Tead-bound, E12.0-accessible (E12.0 ATAC), and E13.5-accessible chromatin regions (E13.5 ATAC). A clear majority (25,423) of the Tead-bound chromatin regions identified by CUT&RUN (C&R) at E12.0, were also identified through ATAC-seq as being accessible at that stage and remained accessible at E13.5, when the progenitor cells exit the cell cycle. (B) Homer motif enrichment analysis confirms a Tead DNA-binding motif is enriched in the Tead-bound chromatin regions that are accessible at E12.0. (C) Identified using GREAT software, gene ontology (GO) terms associated with stem cell maintenance and cell division are enriched in the genes associated with the Tead-bound and ATAC-accessible chromatin regions at E12.0. (D) DeepTools-generated heatmaps demonstrate comparative analysis of the chromatin accessibility, assessed by ATAC-seq (grey), and H3K27Ac (green) of the chromatin regions occupied by Tead in E12.0 progenitor cells. As also demonstrated by Venn diagram in (A), over 85% (25,423) of Tead-occupied accessible chromatin regions identified at E12.0, remain accessible after the cell cycle exit at E13.5. The H3K27Ac status of the same regions remains largely unchanged. (E1-E4) bigWig tracks for the representative examples of the putative Tead target genes associated with cell cycle progression are visualized using Integrative Genomics Viewer (IGV). Note that chromatin associability (ATAC; grey) and H3K27Ac (green) is unchanged at the putative regulatory elements occupied by Tead (blue; black bars). (F) Heatmap showing relative expression levels of the differentially expressed putative Tead targets associated with cell cycle (FDR<0.01; n=2 for each condition). Highly expressed genes are shown in red, while the genes with relatively low levels of expression are depicted in blue. (G) GSEA enrichment plot demonstrates significant correlation (FDR<0.0001) between gene expression and Tead-occupancy for the cell cycle-related genes (GO:0007049) at E12.0.

FIGS. 4A-1, 4A-2, 4A-3, 4A-4, 4A-5, 4B-1, 4B-2, 4C, and 4D. Hippo signaling activation and degradation of Yap protein coincides with the wave of cell cycle exit in the developing organ of Corti. (A1-A3) On the left, the heatmap demonstrates relative expression levels of 30 genes associated with the Hippo pathway in E12.0 and E13.5 OC progenitor cells. Highly expressed genes are shown in red, while genes with relatively low levels of expression are depicted in blue. Differentially expressed genes are highlighted in black (FDR<0.01; n=2 for each condition). On the right, the bar graphs demonstrate FPKM values of some of these up- and down-regulated genes. (B) Schematic depiction of the Hippo pathway. When Hippo signaling is inactive, Yap transcriptional co-factor may translocate to the nucleus where, together with Tead transcription factors, it activates gene expression. Phosphorylation and activation of Mst1/2 and Lats1/2 kinases in the Hippo pathway results in phosphorylation and cytoplasmic retention of Yap, where it is targeted for degradation. Note that most inhibitors of the Hippo pathway (positive regulators of Yap signaling) are highly expressed at E12.0, while most Hippo activators (negative regulators of Yap signaling) are upregulated at E13.5. (C) Western blotting analysis of epithelial cochlear duct lysates comparing Hippo signaling activity at E12.0 and E14.5. Consistent with Hippo signaling activation at E14.5, although the overall levels of Yap protein are unchanged, the levels of the inactive, phosphorylated form of Yap are increased at this stage (n=3 for each condition). (D) To demonstrate the timing of Yap protein degradation compared to the cell cycle exit in the organ of Corti, an EdU pulse was given 30 min prior to sacrificing the pregnant dams at E12.5, E14.5, and neonatal pups at P6. Immunofluorescence analysis show progressive depletion of Yap protein (purple, white) in the Sox2-positive (green and outlined) domain of the cochlear duct as it becomes devoid of EdU-positive (white) proliferating progenitor cells in E12.5 (left panels; n=4)), E14.5 (middle panels; n=4), and P6 (right panels; n=6). Scale bars: 50 m.

FIGS. 5A, 5B, 5C, 5D, 5E, 5F, and 5G. Conditional loss of Yap in the inner ear results in formation of significantly smaller sensory organs. (A) Immunofluorescence analysis shows reduction in number of Ki67-positive (red) cells in the Sox2-positive (green) prosensory domain of the cochlear ducts (outlined) of the Yap CKO (Pax2-CreCre/+ Yapfl/fl) embryos, compared to the WT (Pax2-Cre+/+ Yapfl/fl) littermates at E12.5 (n=9 for each condition). Nuclei are counterstained with DAPI (blue). Scale bars: 100 m. (B) Bar graph demonstrates a significant decrease in proportion of mitotically active Sox2-positive cells in Yap CKO cochlear ducts, compared to WT controls (n=9 for each condition; p=0.0049). (C) Representative immunofluorescence images of the whole mount cochlear ducts of the WT and Yap CKO littermate embryos at E18.5 are labeled for Sox2 (green) to visualize the organ of Corti (n=4 for each condition). (D) The apical and basal turns of WT and Yap CKO cochlea are demonstrated. Supporting cells are labeled for Sox2 (green), hair cells—for Myo7A (red). Ectopic hair cells and supporting cells are seen on the abneural side of the organ of Corti in Yap CKO OC (arrowhead). Scale bars: 100 um. (E) Immunofluorescence images of the sections through the apical turn of the cochlear ducts of the WT and Yap CKO littermate embryos at E18.5. Supporting cells are labeled for Sox2 (green), hair cells—for Myo7A (red). Ectopic hair cells are seen in Yap CKO (asterisks). Nuclei are counterstained with DAPI (blue). Scale bars: 50 m. (F) Bar graphs demonstrate significant decrease of Yap CKO cochlear duct size at E18.5 compared to WT littermates (n=4 for each condition; p<0.0001). (G) Bar graphs demonstrate significant decrease in number of hair cells in the Yap CKO cochlear ducts compared to WT littermates (n=4 for each condition; p=0.0006).

FIGS. 6A, 6B-1, 6B-2, 6C, and 6D. Viral overexpression of Yap5SA in the postnatal inner ear sensory organs in vivo initiates cell cycle re-entry. (A) Schematic representation of the experimental design for diphtheria toxin (DT) hair cell ablation and the following adeno-associated virus (AAV) administration and analysis of the neonatal Pou4f3DTR/+ mice. (B1-B2) Immunofluorescence analysis of P10 whole mount utricles, isolated from the Pou4f3DTR/+ mice in which hair cell ablation was induced at P6 and GFP-control or Yap5SA-GFP AAV injections were performed at P7, is demonstrated. To identify the cells that have re-entered the cell cycle, an EdU pulse was administered at P10, 30 min before sacrificing the animals. Yap5SA overexpression results in a marked increase in cell proliferation (EdU; white) of Sox2-positive (red) supporting cells in the utricular macula. (B′) Same analysis (as in B) is demonstrated for the organs of Corti. (C) Increase in the numbers of proliferating Sox2-positive supporting cells in the utricles, isolated from the Yap5SA-infected mice compared to the GFP-infected controls is statistically significant (n=4 for each condition; p=0.0009). (D) Increase in the numbers of proliferating Sox2-positive cells in cochlea, isolated from the Yap5SA-infected mice compared to the GFP-infected controls is statistically significant (n=6 for each condition; p=0.032) compared to GFP-infected littermates. Nuclei are counterstained with DAPI (blue). Scale bars: 100 m.

FIGS. 7A, 7B, 7C, and 7D. Conditional loss of Yap in the inner ear results in significant reduction of the number of Sox2-positive progenitor cells and loss of cell proliferation in the outer sulcus. (A) Immunofluorescence analysis demonstrates no active caspase 3 labeling (red) in Sox2-positive (green) prosensory domains of the E12.5 cochlear ducts of either WT (Pax2-Cre+/+ Yapfl/fl) or Yap CKO (Pax2-CreCre/+ Yapfl/fl) littermates (n=6 for each condition). Nuclei are counterstained with DAPI (blue). Scale bars: 100 m. (B) The number of Sox2-positive cells is reduced significantly in Yap CKO cochlea compared to WT (n=9 for each condition; p=0.024). (C) Immunofluorescence analysis shows depletion of Yap protein (magenta) in the cochlear ducts (outlined) of the Yap CKO embryos, compared to the WT littermates at E13.5 (n=4 for each condition). In the WT organ of Corti, cells on the abneural convex side lateral from p27Kip1-positive (green) domain are actively dividing (EdU in white; arrows). In contrast, in the Yap CKO organ of Corti, the p27Kip1-positive domain is expanded at the apex and devoid of EdU-positive cells. EdU pulse was given 30 min prior to the analysis. Scale bars: 100 m. (D) Bar graph demonstrates that decrease in cell proliferation in the abneural domain of the Yap CKO cochlear ducts is statistically significant (n=4 for each condition; p=0.0056).

FIGS. 8A, 8B, 8C, 8D, and 8E. Conditional loss of Yap in the otic progenitor cells results in significant reduction of the vestibular sensory organ size. (A) Compared to the WT littermates (Pax2-Cre+/+ Yapfl/f), Yap CKO (Pax2-CreCre/+ Yapfl/fl) embryos develop exencephaly by E18.5 and die shortly afterbirth. (B) Immunofluorescence analysis of the vestibular sensory organs of the Yap CKO and WT littermate embryos at E18.5 demonstrates reduction of the utricular and saccular sensory epithelia, in which cells are labeled with Sox2 (green). Scale bars: 100 m. (C) The areal size of the utricular macula is significantly reduced in the Yap CKO embryos compared to WT controls at E18.5 (n=5 for WT and n=4 for Yap CKO; p<0.0001). (D) Similarly, the areal size of the of the saccular macula is also significantly reduced in the Yap CKO embryos compared to WT controls at E18.5 (n=3 for WT and n=5 for Yap CKO; p<0.0001). (E) Hair cell differentiation is unaffected in absence of Yap, as hair cell density is unchanged in the vestibular sensory epithelia of Yap CKO embryos compared to WT controls at E18.5 (n=4 for WT and n=5 for Yap CKO; p=0.7).

FIGS. 9A, 9B-1, 9B-2, and 9B-3. Lateral ventricle injections of Anc80 viral vectors is an effective method for genetic manipulation in the inner ear. (A) Schematic representation of the endolymphatic (blue) and perilypmphatic (purple) fluid compartments of the inner ear and their connection to the cerebrospinal fluid (CSF) via cochlear aqueduct is demonstrated. (B) Immunofluorescence analysis of the sections and the whole mount inner ear sensory organs dissected from P6 mice injected into the lateral ventricle with 5 ul of Anc80-CMV-GFP viral vector 48 hr prior, demonstrates widespread GFP expression (green) in cerebellum, utricle, and in the organ of Corti, including hair cells (Myo7a; blue). Nuclei are counterstained with DAPI (blue). Scale bars: 100 μm. (C) Schematic representation of the experimental design for hair cell ablation via Diphtheria toxin injections (DT) and viral gene transfer in Pou4f3DTR/+ mice. (D) Immunofluorescence analysis of the inner ears of the Pou4f3+/+(WT controls) and Pou4f3DTR/+ mice. At postnatal day 6, 3 days after DT injection, widespread hair cell damage and death is observed in the Pou4f3DTR/+ mice compared to the WT littermates. In undamaged ears, GFP expression (green), induced by Anc80-CMV-GFP injection into the lateral ventricle at P4, is only seen in the hair cells (Myo7a; Blue). In contrast, after hair cell damage, GFP expression (green) is observed in the residual supporting cells, intercalating dying sensory receptors. Scale bars: 25 μm.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “about” means that the amount or value in question may be the specific value designated or some other value in its neighborhood. Generally, the term “about” denoting a certain value is intended to denote a range within +/−5% of the value. As one example, the phrase “about 100” denotes a range of 100+/−5, i.e. the range from 95 to 105. Generally, when the term “about” is used, it can be expected that similar results or effects according to the invention can be obtained within a range of +/−5% of the indicated value.

As used herein, the term “and/or” means that either all or only one of the elements of said group may be present. For example, “A and/or B” shall mean “only A, or only B, or both A and B”. In the case of “only A”, the term also covers the possibility that B is absent, i.e. “only A, but not B”.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The phrase “composed of” means “including” or “consisting of.” Typically, this phrase is used to denote that an object is formed from a material.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within +0%, 0.1%, 0.5%, 1%, 2%, 3%, 4%, 5% or 10% of the value or relative characteristic.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

Throughout this application, where publications are referenced, the disclosures of these publications in their entireties are hereby incorporated by reference into this application to more fully describe the state of the art to which this invention pertains.

### Abbreviations

“AAV” means Adeno-associated virus (AAV).

“ITR” means inverted terminal repeat.

“PGH pA” means bovine growth hormone polyA signal.

“WPRE” means woodchuck hepatitis virus posttranscriptional regulatory element.

“WT” means wild type.

“YAP” means yes-associated protein.

The term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

The term “expression control sequence” refers to a nucleic acid sequence that regulates the expression of a nucleotide sequence to which it is operably linked. An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons. The term “expression control sequence” is intended to include, at a minimum, a sequence whose presence are designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, sequences referred to as polyA sequences. It also can be designed to enhance mRNA stability. Expression control sequences which affect the transcription and translation stability, e.g., promoters, as well as sequences which effect the translation, e.g., Kozak sequences, are known in insect cells. Expression control sequences can be of such nature as to modulate the nucleotide sequence to which it is operably linked such that lower expression levels or higher expression levels are achieved.

The term “promoter” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer. A “tissue specific” promoter is only active in specific types of tissues or cells.

The term “heterologous promoter” refers to a promoter that does not naturally direct expression of the promoter in nature.

The term “natural promoter” refers to a promoter found in nature with the YAP gene.

The term “expression vector” means a construct designed for gene expression in cells. Expression vectors includes but are not limited to, virus, plasmids, cosmid, transposons, and the like.

The terms “percent identical” or “percent identity” refer to nucleic acid or amino acid sequences that are substantially identical to a coding sequence or amino acid sequence for the constitutively active Yap5SA gene (SEQ ID NO: 1) or amino acid sequence thereof (SEQ ID NO: 6) or the wildtype YAP gene with at least a 127SA mutation (SEQ ID NO: 2) or amino acid sequence thereof (SEQ ID NO: 7).

The term “substantially identical” means nucleotide sequence with similarity to the nucleotide sequence of the constitutively active Yap5SA gene (SEQ ID NO: 1) or the wildtype YAP gene sequence with at least a 127SA mutation (SEQ ID NO:2). The term “substantially identical” can also be used to describe similarity of polypeptide sequences. For example, nucleotide sequences or polypeptide sequences that are at least 70%, 75%, 80%, 85%, 90%, 92%, 95%, 96%, 98% or 99% identical to the wildtype YAP gene sequence with at least a 127SA mutation (SEQ ID NO:2) or the constitutively active Yap5SA gene (SEQ ID NO: 1) coding sequences, or the encoded polypeptides thereof, respectively, or fragments or derivatives thereof, and still retain ability to activate Yap/Tead signaling in a subject.

To determine the “percent identity” (i.e., percent sequence identity) of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a refinement, the sequences are aligned for maximum correspondence over a specified comparison window, as measured by sequence comparison algorithms or by visual inspection. In a refinement, the length of a first sequence aligned for comparison purposes is at least 80% of the length of a second sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5. In this regard, the following oligonucleotide alignment algorithms may be used: BLAST (GenBank URL: www.ncbi.nlm.nih.gov/cgi-bin/BLAST/, using default parameters: Program: BLASTN; Database: nr; Expect 10; filter: default; Alignment: pairwise; Query genetic Codes: Standard(1)), BLAST2 (EMBL URL: http://www.embl-heidelberg.de/Services/index.html using default parameters: Matrix BLOSUM62; Filter: default, echofilter: on, Expect: 10, cutoff: default; Strand: both; Descriptions: 50, Alignments: 50), or FASTA, search, using default parameters. When sequences differ in conservative substitutions, the percent identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity.

Nucleotide sequences encoding the constitutively active YAP gene of the invention may also be defined by their capability to hybridize with the nucleotide sequence of SEQ ID NO:1 or SEQ ID NO: 2 or complementary sequences SEQ ID NO:4 and SEQ ID NO:5 respectively thereof, under stringent hybridization conditions. Stringent hybridization conditions are herein defined as conditions that allow a nucleic acid sequence of at least about 25, preferably about 50 nucleotides, 75 or 100 and most preferably of about 200 or more nucleotides, to hybridize at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridization is for at least 10 hours and preferably washing is performed for at least one hour with at least two changes of the washing solution. These conditions will usually allow the specific hybridization of sequences having about 90% or more sequence identity.

In an embodiment, a method for inducing sensory receptor regeneration is provided. The method includes a step of identifying a subject in need of regeneration of inner ear sensory epithelia. This identification can be accomplished by hearing tests and balance tests. The method also includes a step of activating Yap/Tead signaling in the subject. Advantageously, the method can be used as a therapy for hearing loss and/or balance disorders.

In one variation, Yap/Tead signaling is activated by introducing an expression vector encoding constitutively active YAP gene into the subject such that the expression vector contacts inner ear sensory epithelia in a sufficient amount to induce regeneration thereof. In this context, the constitutively active YAP gene is an open reading frame or fragment thereof. In one refinement, the expression vector is introduced by injection into a subject's round window or posterior semicircular canal. In a refinement, the expression vector is a viral vector. Examples of virus that can be used as expression vectors include, but are not limited to, adenoviruses, adeno-associated viruses, retroviruses, lentiviruses, vaccinia viruses, vesicular stomatitis virus, herpes viruses, maraba virus, or papilloma viruses. One particularly useful, viral vectors are AAV vectors such an Anc80 virus.

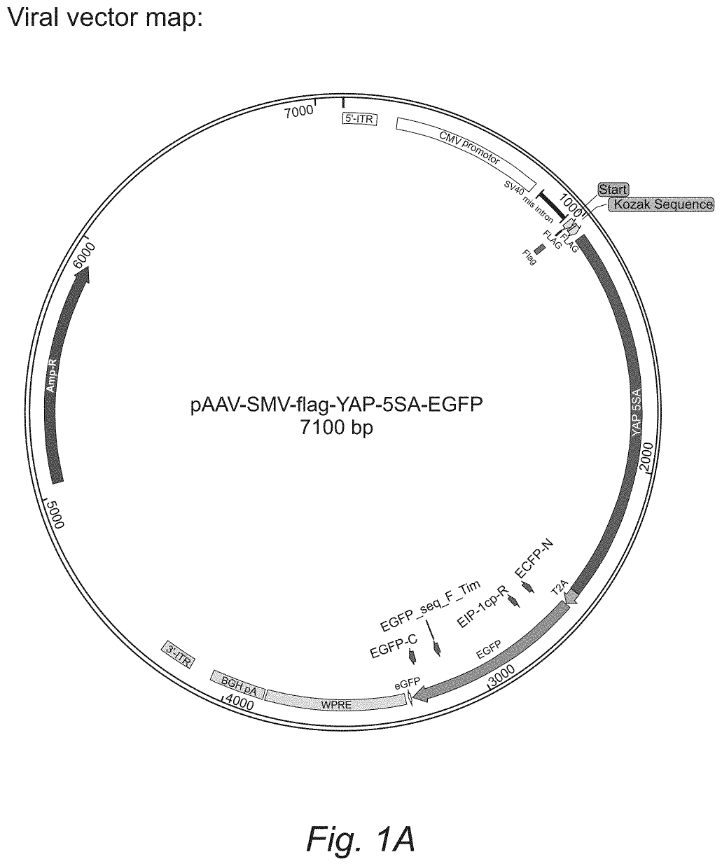

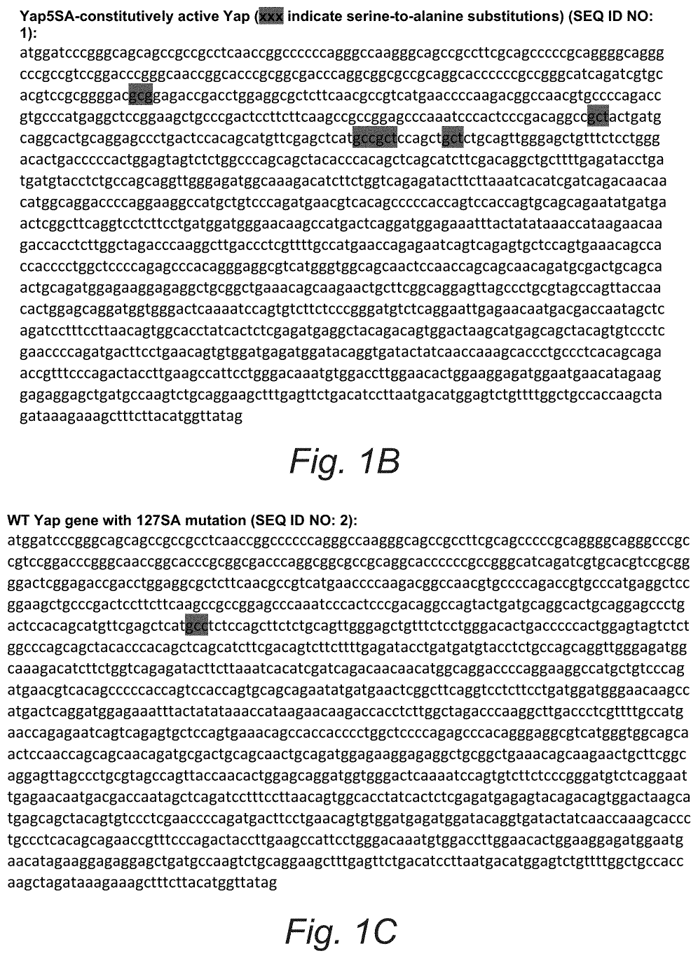

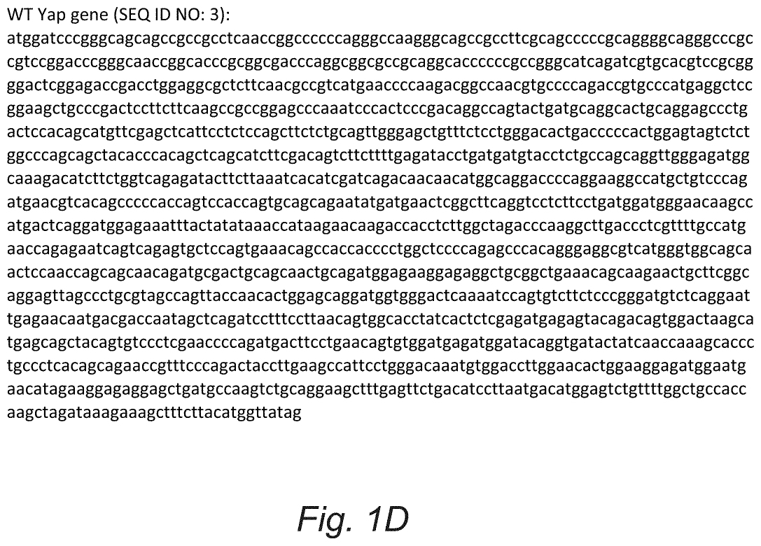

FIG. 1A provides a vector map of the pAAV-CMC-flag-YAP-5SA-EGFP plasmid used in the experiments below. The YAP SSA encoding polynucleotide segment is positioned downstream (in the direction of transcription). Downstream of the YAP SSA encoding polynucleotide is a green fluorescent protein encoding polynucleotide. The green fluorescent protein encoding polynucleotide segment would not be present in a vector for treating a subject. WPRE encoding polynucleotide segment is downstream of the YAP SSA encoding polynucleotide segment and the green fluorescent protein encoding segment. PGH pA for polyadenylation is downstream of the WPRE encoding polynucleotide segment. A 3′-ITR is downstream of the PGH pA. FIG. 1B provides a polynucleotide sequence encoding YAP5SA which is constitutively active YAP (SEQ ID NO: 1). The polypeptide sequence corresponding to SEQ ID NO: 1 is SEQ ID NO: 6. This constitutively active YAP gene includes the following serine to alanine mutations: 61SA, 109SA, 127SA, 128SA, and 131SA. (Mutations are indicated by the amino acid position in the corresponding protein, see SEQ ID Nos: 6 and 7). Additionally, serines at positions 163, 164, and 381 can be changed to Alanine to increase Yap activity. FIG. 1C provides a polynucleotide sequence encoding WT YAP gene with 127SA mutation (SEQ ID NO: 2). The polypeptide sequence corresponding to SEQ ID NO: 2 is SEQ ID NO: 7. FIG. 1D provides a polynucleotide sequence encoding WT YAP gene. (SEQ ID NO: 3).

Typically, the expression vector includes expression control sequence operably linked to a nucleotide sequence encoding a constitutively active YAP gene. For example, the Yap5SA gene used in the experiments below is a constitutively active YAP gene. In a variation, the constitutively active YAP gene is substantially identical to the Yap5SA gene. In a refinement, the constitutively active YAP gene that is substantially identical to the Yap5SA gene is a nucleotide sequence that is at least 70%, 75%, 80%, 85%, 90%, 92%, 95%, 96%, 98% or 99% identical to SEQ ID NO: 1 (i.e., the constitutively active Yap5SA open reading frame) while retaining the ability to activate Yap/Tead signaling in a subject. Therefore, the constitutively active YAP gene maintains one or more of the serine to alanine mutations in SEQ ID NO: 1. In a refinement, the constitutively active YAP gene maintains any 1, 2, 3, or 4 of the serine to alanine mutations in SEQ ID NO: 1. In a further refinement, the constitutively active YAP gene maintains all 5 of the serine to alanine mutations in SEQ ID NO: 1.

In another variation, the constitutively active YAP gene is a nucleotide sequence that is at least 70%, 75%, 80%, 85%, 90%, 92%, 95%, 96%, 98% or 99% identical to the SEQ ID NO:2 (i.e., the wildtype YAP gene sequence with at least a 127SA mutation) while retaining the ability to activate Yap/Tead signaling in a subject. In a refinement, the constitutively active YAP gene that is identical to SEQ ID NO: 1 with the provided percentages maintains the 127SA mutation.

In another variation, the constitutively active YAP gene is a nucleotide sequence of the wild type YAP gene having SEQ ID NO:3 with at least one of the following mutations: 61SA, 109SA, 127SA, 128SA, 131SA, 163SA, 164SA, and 381SA. In another variation, the constitutively active YAP gene is a nucleotide sequence of the wild type YAP gene having SEQ ID NO:3 with any 1, 2, 3, 4, 5, 6, 6, or all of the following mutations: 61SA, 109SA, 127SA, 128SA, 131SA, 163SA, 164SA, and 381SA.

In another variation, the constitutively active YAP gene is a nucleotide sequence having accession numbers NM_001130145, NM_001195044, NM_001195045, NM_001282097, or NM_001282098. In a refinement, the constitutively active YAP gene is a nucleotide sequence that is at least 70%, 75%, 80%, 85%, 90%, 92%, 95%, 96%, 98% or 99% identical to a nucleotide sequence having accession numbers NM_001130145, NM_001195044, NM_001195045, NM_001282097, or NM_001282098 while retaining the ability to activate Yap/Tead signaling in a subject. In a further refinement, the constitutively active YAP gene is a nucleotide sequence having the identities percentages set forth above with respect to a nucleotide sequence having accession numbers NM_001130145, NM_001195044, NM_001195045, NM_001282097, or NM_001282098 with any 1, 2, 3, 4, 5, 6, 6, or all of the following mutations: 61SA, 109SA, 127SA, 128SA, 131SA, 163SA, 164SA, and 381SA.

As set forth above, the polypeptide sequence corresponding to SEQ ID NO: 1 is SEQ ID NO: 6 and the polypeptide sequence corresponding to SEQ ID NO: 2 is SEQ ID NO: 7. In a refinement, the constitutively active YAP gene is a nucleotide sequence encoding polypeptides having sequences SEQ ID NO: 6 or SEQ ID NO: 7 with 1, 2, 3, 4, 5, 6, 7, 8, 9 or 10 conservative substitutions. The conservative substitutions are similar to the amino acid be changed with respect to polarity, charge, solubility, hydrophobicity, hydrophilicity, and/or the amphipathic nature of the residues, while preserving the functionality of being constitutively active. Conservative substitutions that may be made are, for example, substitutions between aliphatic amino acids (alanine, valine, leucine, isoleucine), polar amino acids (glutamine, asparagine, serine, threonine), acidic amino acids (glutamic acid and aspartic acid), basic amino acids (arginine, lysine and histidine), aromatic amino acids (phenylalanine, tryptophan and tyrosine), large amino acids (phenylalanine and tryptophan), small amino acids (glycine, alanine) and hydroxyl amino acids (serine, threonine).

In a refinement, the expression of the YAP gene can be directed by the YAP gene's natural promoter or by a heterologous promoter. Examples of suitable heterologous promoters include but are not limited to a cytomegalovirus promoter, a chicken beta actin promoter, a synthetic CASI promoter, a phosphoglycerate kinase promoter, and an elongation factor (EF)-1 promoter, an alpha9 nicotinic receptor promoter, a prestin promoter, a growth factor independent (GFI1) promoter, a vesicular glutamate transporter 3 (VGLUT3) promoter, and Glial fibrillary acidic protein (GFAP) promoter.

The techniques for packaging a constitutively active YAP gene into a virus are well known. In a refinement, a first construct that includes a nucleic acid sequence encoding a capsid protein (e.g., Anc80 capsid protein) and a second construct carrying the constitutively active YAP gene are utilized and allows for the constitutively active YAP gene to be packaged within the Anc80 capsid protein. In a further refinement, the constitutively active YAP gene is flanked by suitable Inverted Terminal Repeats (ITRs) are provided.

The constitutively active YAP gene can be packaged in a virus by using a packaging host cell such as HEK 293T cells. Viral components can be introduced into the packaging host cell using one or more constructs including the first and second construct set forth above. Examples of viral components include, but are not limited to, rep sequences, cap sequences, inverted terminal repeat (ITR) sequences and other components know to those skilled in the art. In a refinement, the viruses set forth herein in include a capsid protein, and, in particular, an Anc80 capsid protein. In a further refinement, the pAnc80L65AAP plasmid is packaged into a packaging host cell. The pAnc80L65AAP plasmid includes an adeno-associated Viral Vector (AAV) capsid Anc80L65 in AAV2Rep expression construct with endogenous AAP.

In another variation, Yap/Tead signaling is activated by inhibiting or activating the upstream regulators. In a refinement, inhibiting or downregulating expression of one or more of Lats1/2, Mst1/2, Sav1, Mob1a/b, Nf2, Wwc1, Vgll4, or Dchs1 (i.e., proteins, RNA, or genes thereof) is used to activate Yap/Tead signaling. In another refinement, expression of one or more of Lats1/2, Mst1/2, Sav1, Mob1a/b, Nf2, Wwc1, Vgll4, or Dchs1 is inhibited or downregulated by targeting gene expression (CRISPR) or RNA translation (siRNA, shRNA). In another refinement, expression of one or more of Dlg5, Ajuba, Wtip, or Tead1-4 is activated or upregulated to activate Yap/Tead signaling.

In another embodiment, a pharmaceutical composition includes for activating Yap/Tead signaling in a subject. The pharmaceutical composition includes a pharmaceutically acceptable carrier liquid and an expression vector encoding a constitutively active YAP gene as set forth above. The expression vector is dispersed in the pharmaceutically acceptable carrier liquid at a sufficient concentration to deliver a pharmaceutically effective amount to the subject. Examples of pharmaceutically acceptable carrier liquid include water and saline. The pharmaceutical composition may also include one or more stabilizing additives (e.g., to prevent crystallization) and/or buffers.

Additional details about the invention are set forth in K. Gnedeva, Organ of Corti size is governed by Yap Tead-mediated progenitor self-renewal, PNAS Jun. 16, 2020 117 (24) 13552-13561; first published Jun. 1, 2020; https://doi.org/10.1073/pnas.2000175117, and the associated supporting information; the entire disclosure of these publications are hereby incorporated by reference in their entirety.

The following examples illustrate the various embodiments of the present invention. Those skilled in the art will recognize many variations that are within the spirit of the present invention and scope of the claims.

Results

A Self-Renewal Gene Network is Rapidly Repressed in Organ of Corti Progenitor Cells Between E12 and E13.5.

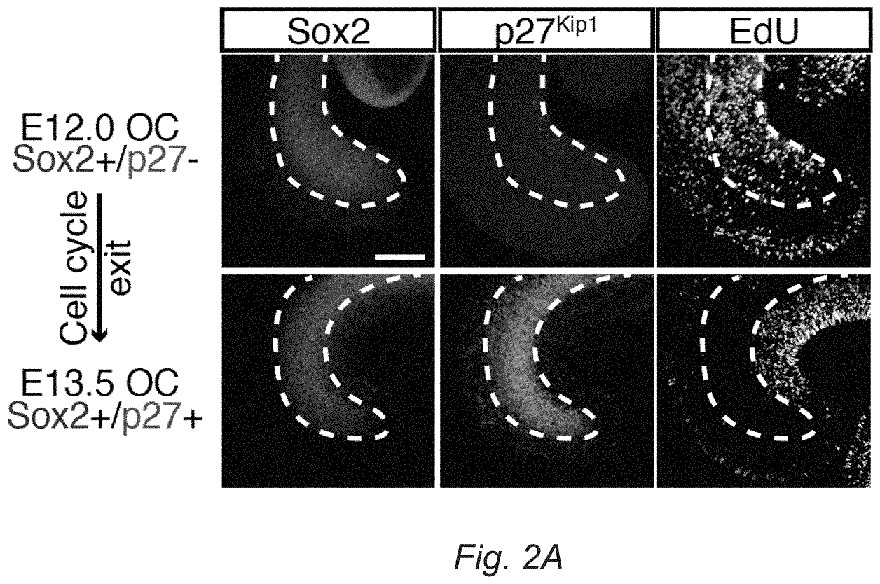

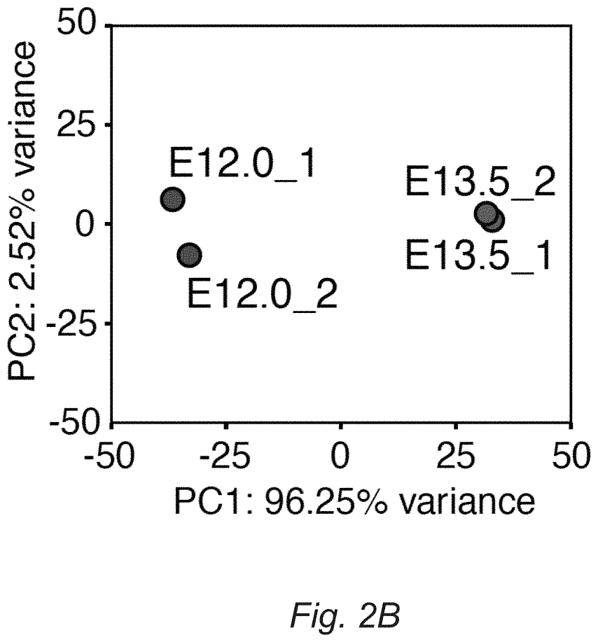

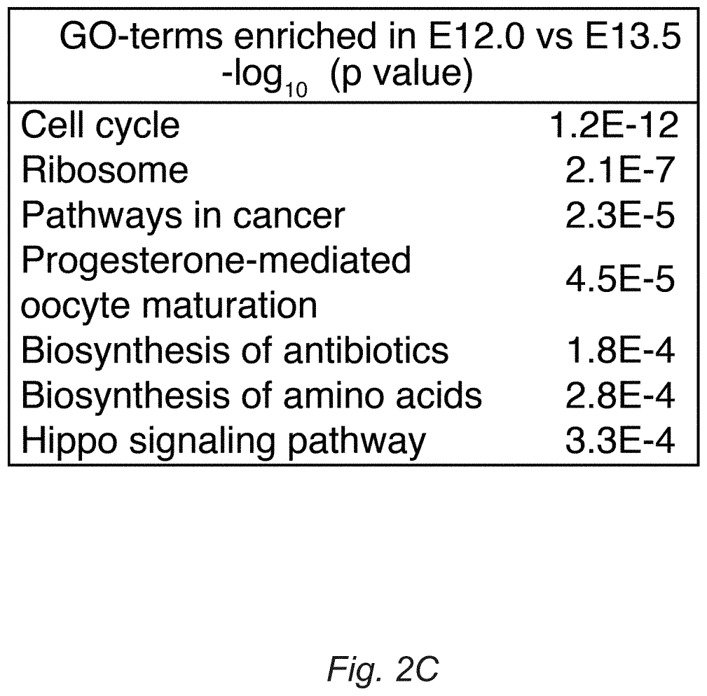

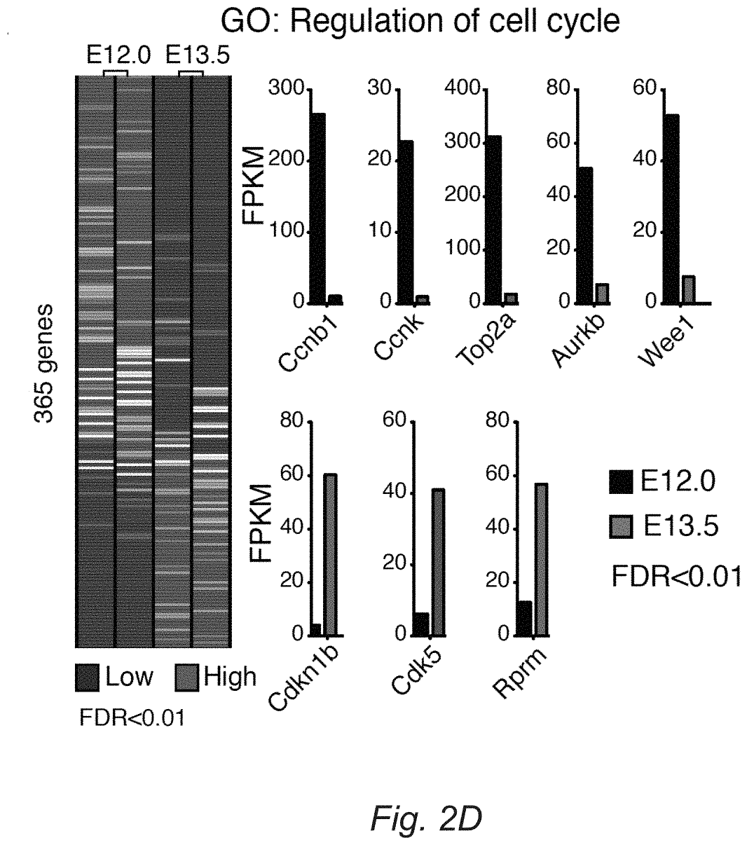

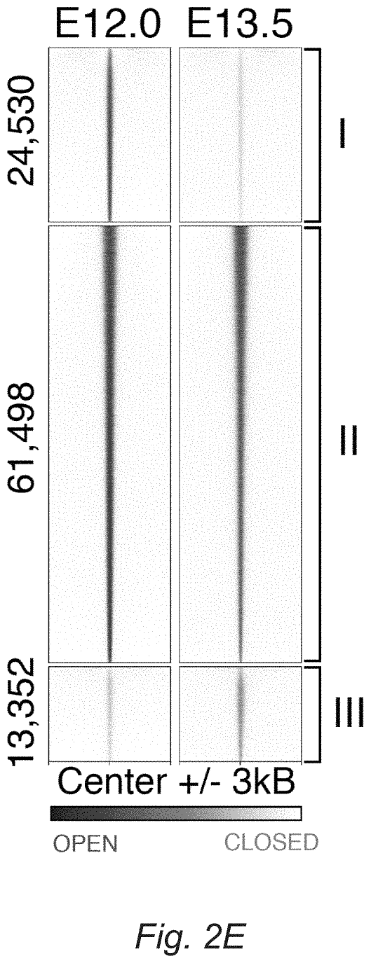

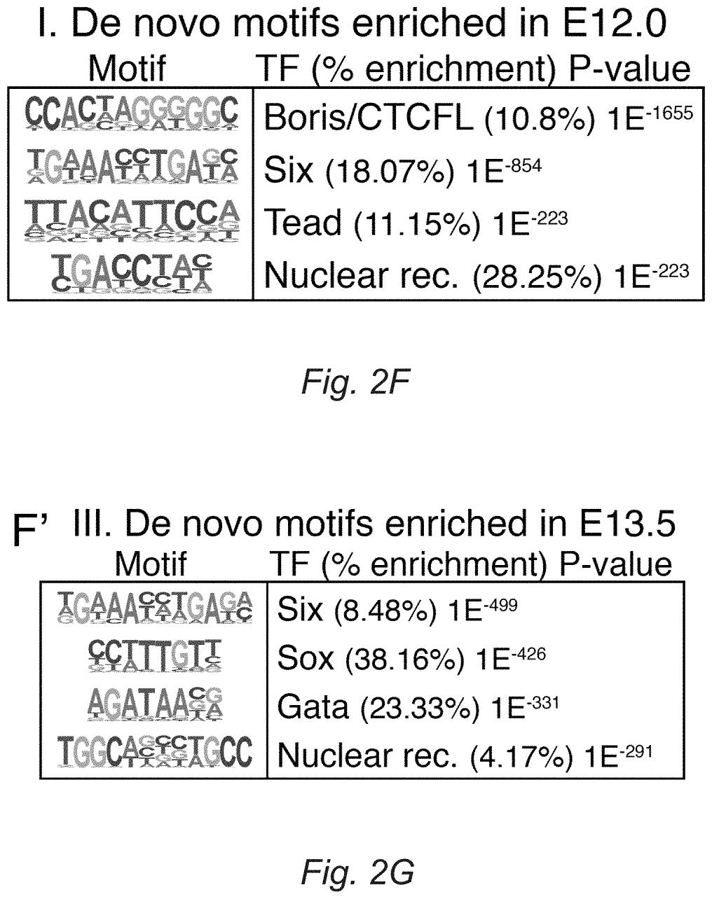

To identify the gene network that controls self-renewal in the developing organ of Corti, gene expression in actively dividing (E12.0) and post-mitotic (E13.5) progenitor cells was analyzed. We used Sox2-GFP mice (17) to purify progenitors at E12.0, and p27^{Kip1}-GFP mice (8) to purify progenitor cells at E13.5 (FIG. 2A). Principal component analysis of RNA-sequencing data revealed that the overwhelming percentage of variance (96%) between E12.0 and E13.5 samples could be explained by the first principal component, comprised of genes associated with cell division (FIG. 2B,C). In particular, 365 genes shown to be associated with regulation of the cell cycle (GO:0051726) were significantly differentially expressed between the two time points, facilitating a sharp transition to a post-mitotic state (FIG. 2D; FDR<0.01). These genes included known key regulators of cell proliferation in the developing cochlea, such as cyclin D1, (Ccnd1)(10) and p27^{Kip1} (Cdkn1b) (7)(9).

Tead Transcription Factors Control Self-Renewal Gene Network in the Organ of Corti Progenitor Cells.

To gain a mechanistic understanding of how proliferation in the cochlear prosensory domain is controlled prior to cell cycle exit, the presumptive regulatory elements specific for the self-renewal state was identified. By profiling chromatin accessibility in E12.0 and E13.5 organ of Corti progenitor cells using ATAC-sequencing, it was demonstrated that over two thirds of all accessible chromatin regions identified in E12.0 progenitor cells remained open as these cells exited the cell cycle, while the one-third of regions were specifically associated with the self-renewal state (FIG. 2E). Transcription factor motif enrichment analysis, using Homer software (18), demonstrated that Tead DNA-binding motifs were among the most significantly enriched in accessible chromatin regions specific to E12.0 progenitors, and regions common to E12.0 and E13.5 progenitors, but not in accessible chromatin regions seen only in E13.5 progenitors (FIG. 2F,G).

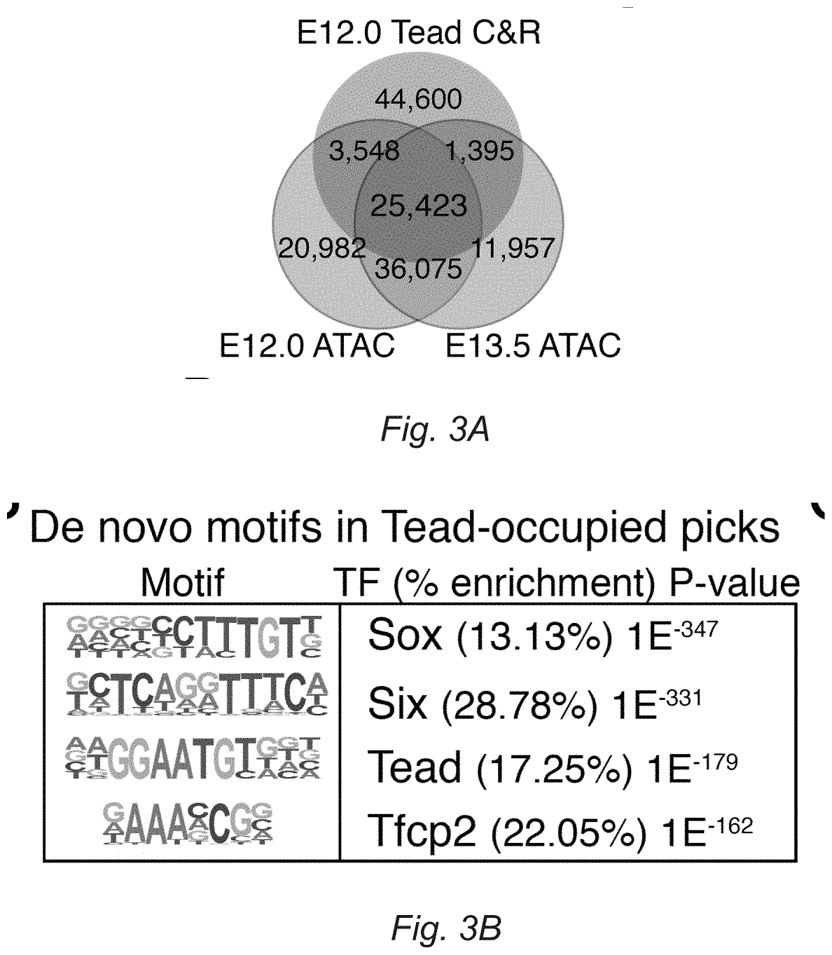

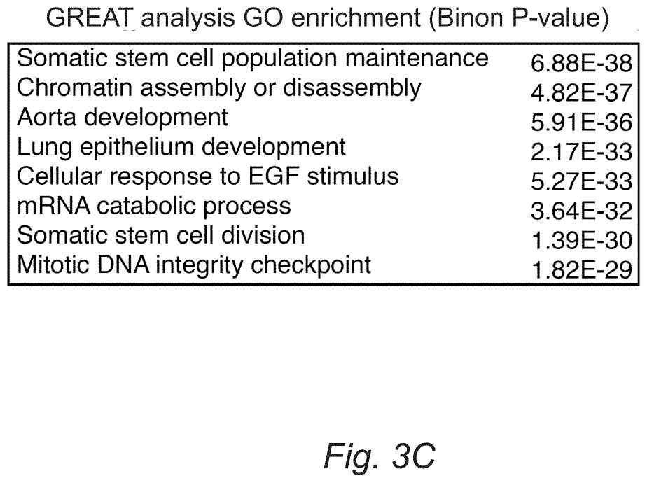

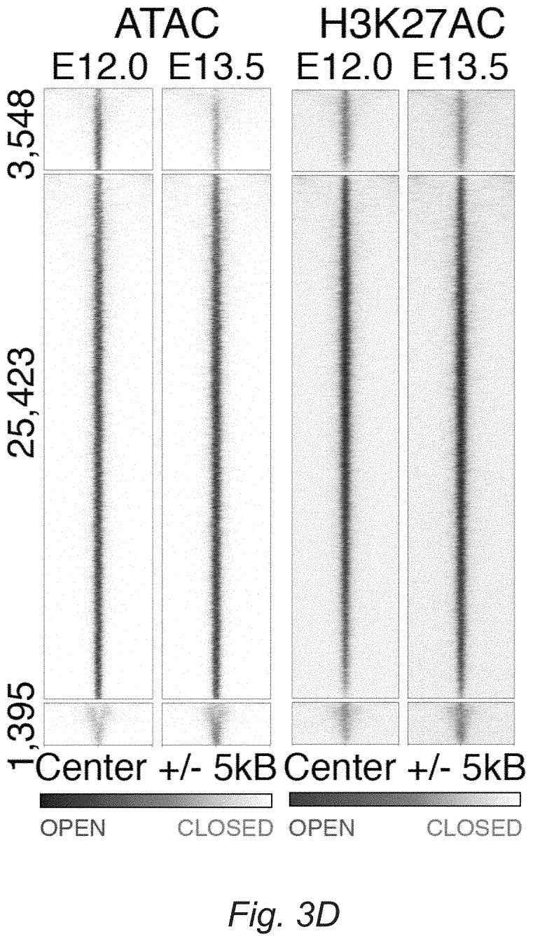

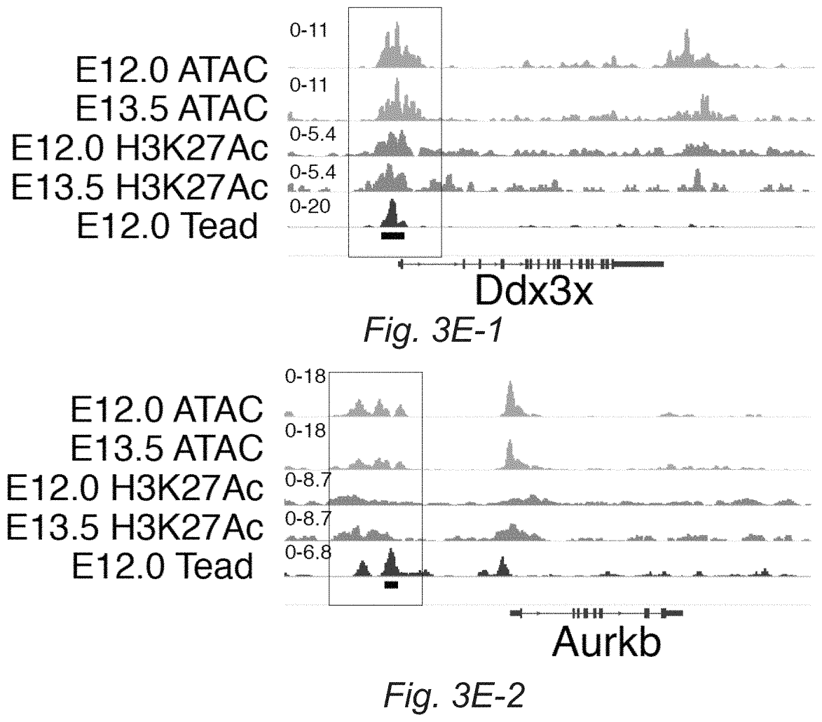

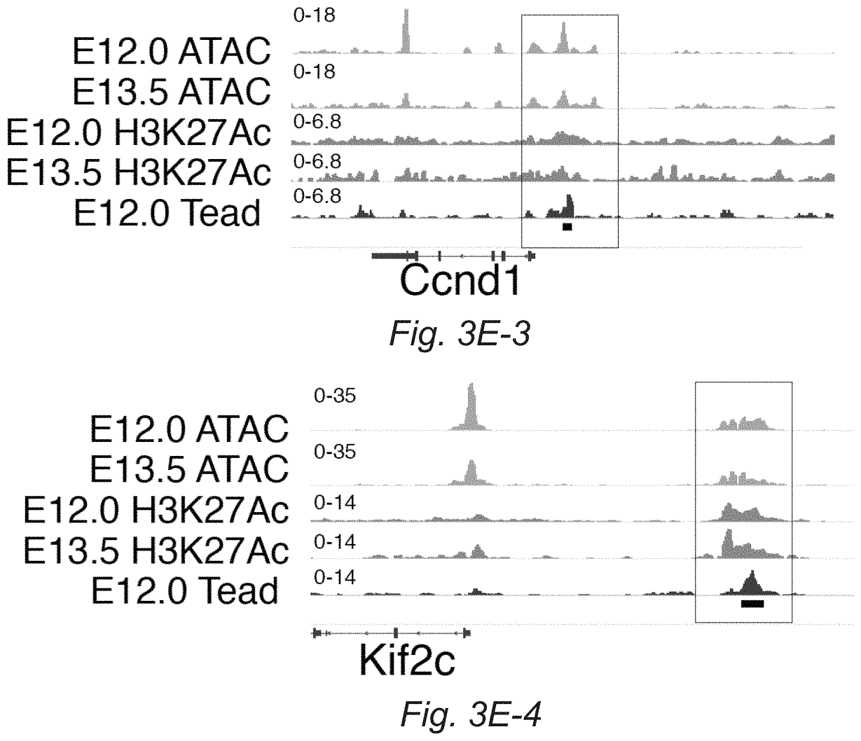

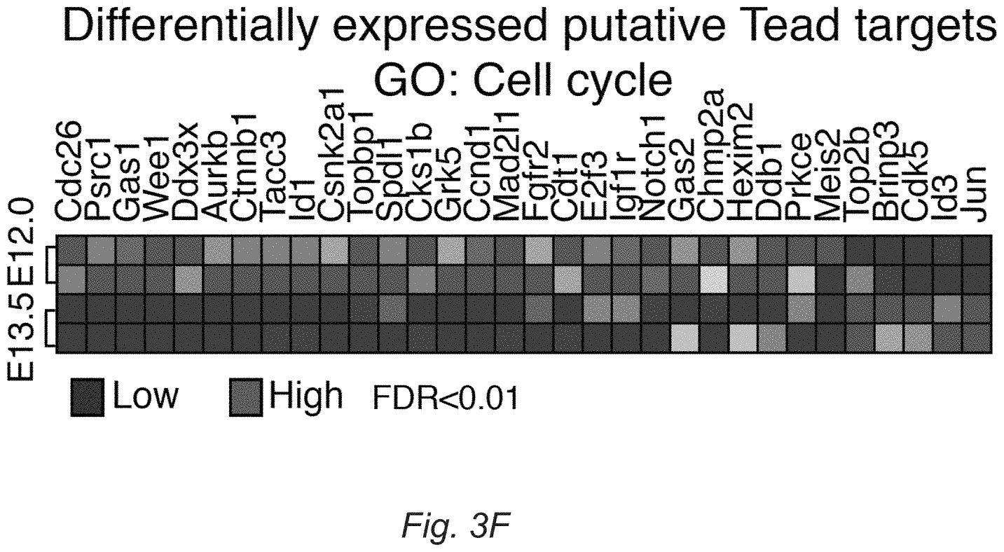

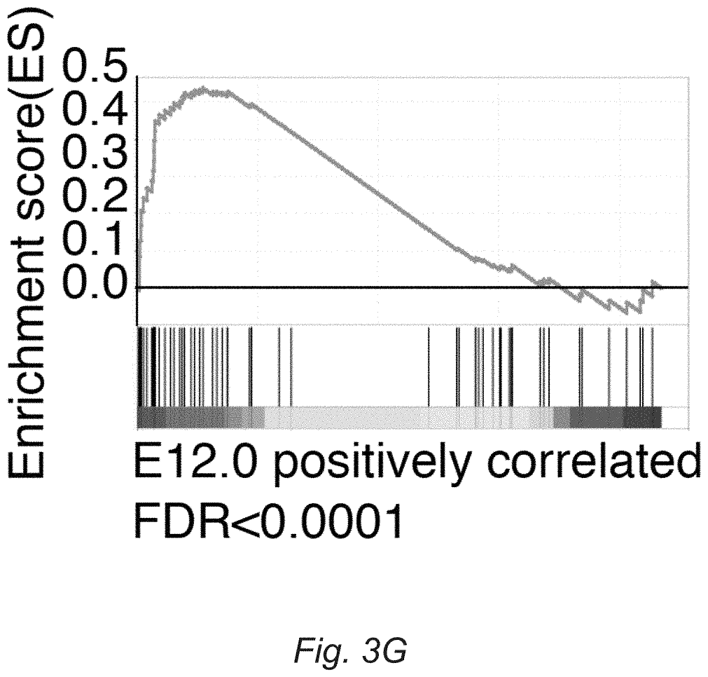

Using a recently published low-input in situ alternative to Chip-sequencing, CUT&RUN (19)(20), it was tested whether Tead transcription factors bound directly to the regulatory elements associated with the proliferative state in the E12.0 organ of Corti. The analysis identified 74,966 chromatin regions occupied by Tead inclusive of two CUT&RUN replicates, almost 40% of which (28,648) mapped to the open chromatin regions identified by ATAC-seq at the same stage (FIG. 3A). A CUT&RUN for histone 3 lysine 27 acetylation (H3K27Ac), a known marker of active promoters and enhancers, was also performed (21)(22). Strikingly, over 85% (24,845) of Tead-bound accessible chromatin regions were also marked by H3K27Ac, suggesting these regions were active regulatory elements in E12.0 progenitor cells. GREAT analysis (23) revealed that terms associated with stem cell maintenance and cell division were among the most enriched in the genes closest to, and thus likely to be controlled by (22) (24), Tead-bound putative regulatory elements (FIG. 3C).

Chromatin accessibility and H3K27Ac status of most (>85%) putative regulatory elements bound by Tead in E12.0 progenitors remained unchanged as these cells exited the cell cycle (FIG. 3D, E). Nevertheless, the putative Tead targets included many positive regulators of the cell cycle, that were downregulated between E12.0 and E13.5 (FIG. 3F). Examples of such regulators included ATP-dependent RNA helicase (Ddx3x) (25), Aurora B kinase (Aurkb) (26), Cyclin d1(Ccnd1) (27), mitotic centromere-associated kinase (Kif2c) (28) and many others (FIG. 3E). Gene Set Enrichment Analysis (GSEA) (29) confirmed that putative Tead target genes associated with the cell cycle (GO:0007049) included almost none of the negative regulators, and thus were significantly coordinately downregulated in the cochlear progenitors between E12.0 and E13.5 (FIG. 3G). These data strongly suggest that Tead transcription factors directly control the self-renewal gene network in the developing organ of Corti prior to the cell cycle exit.

Degradation of Yap Protein is Associated with Cell Cycle Exit in the Organ of Corti.

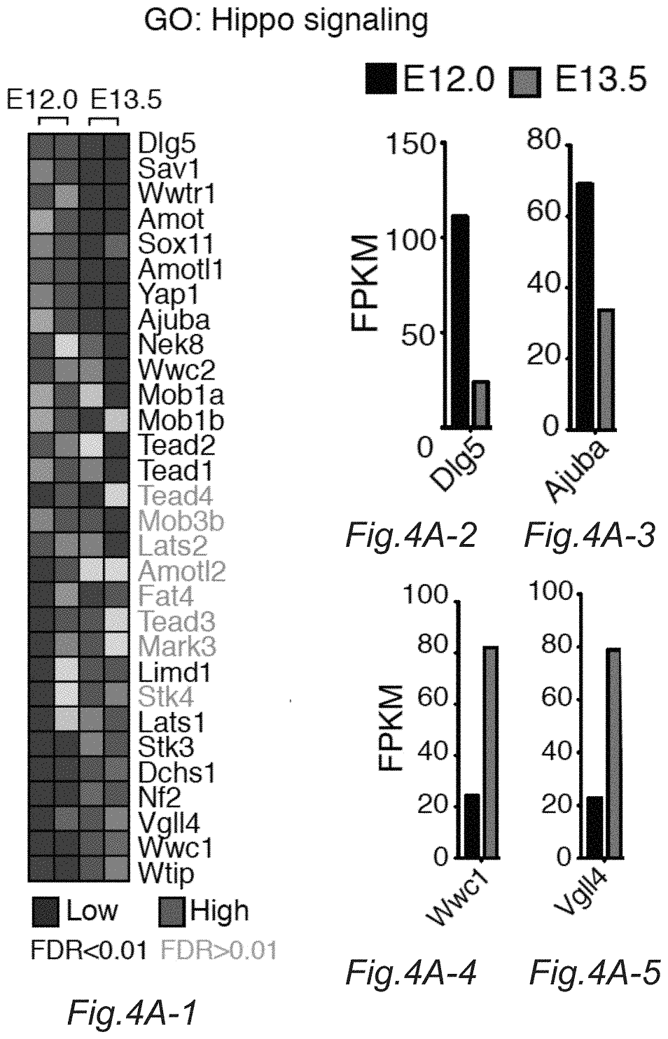

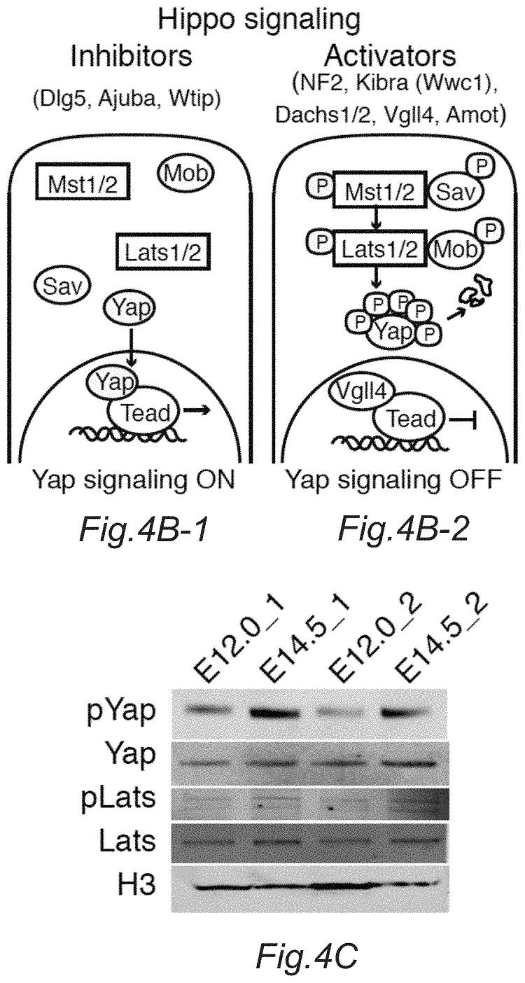

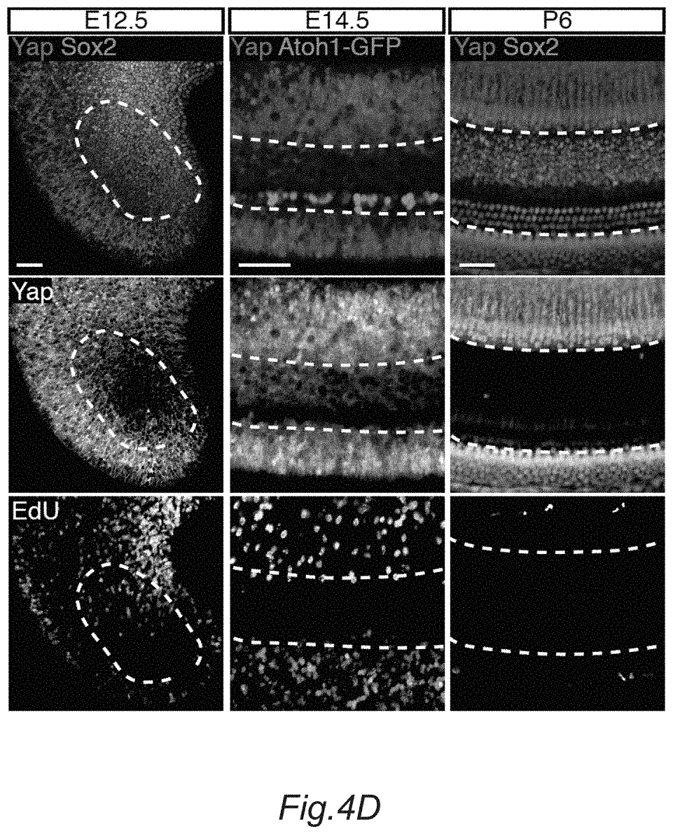

It is well-established that Tead transcription factors activate gene expression in a complex with Yap and Taz co-factors—the downstream effectors of the Hippo signaling pathway (12) (FIG. 4B). Gene ontology analysis demonstrated that Hippo signaling was one of the most enriched terms among the genes differentially expressed between E12.0 and E13.5 (FIG. 4C). Of 30 genes currently associated with Hippo Signaling (GO:0035329), expression of 22 was significantly changed in sensory progenitor cells during cell cycle exit (FDR<0.01; FIG. 4A). Most notably, the transcriptional activators Yap and Wwtr1 (Taz), were downregulated over 2 fold, while Dlg5, a known suppressor of the Hippo signaling pathway that inhibits the association between Mst1/2 and Lats1/2 kinases (30), was downregulated over 5-fold between E12.0 and E13.5. Additionally, Mst2, Lats1, Nf2, Vgll4, and Wwc1(Kibra) were all significantly upregulated in post-mitotic progenitor cells, consistent with activation of Hippo signaling (31).

Because the level of gene expression does not directly correlate with Yap activity, we investigated the phosphorylation state of the key proteins in the Hippo pathway in the actively dividing and post-mitotic organ of Corti. The wave of cell cycle exit initiated at the apex at E12.5, reaches the base of the cochlea by E14.5, thus these two time points were chosen for the analysis (7). We demonstrated that, although the total amount of Yap protein remained relatively unchanged between E12.5 and 14.5, the level of Yap phosphorylation increased between these stages, suggesting activation of Hippo signaling at E14.5 (FIG. 4C).

In addition to phosphorylation status, nuclear versus cytoplasmic localization of Yap serves as a proxy for its activity (31) (FIG. 4B), and thus, we focused on Yap protein distribution during normal organ of Corti development. At E12.5, when the first progenitor cells at the apex of the prosensory domain of the cochlear duct begin to exit the cell cycle, cytoplasmic retention and some degradation of Yap protein was observed (FIG. 4D). As the wave of cell cycle exit progresses and reaches the base of the cochlea by E14.5, the Sox2-positive domain, in which the first Atoh1-positive sensory cell differentiation occurs, can be clearly identified as a Yap protein-depleted region where little to no nuclear Yap protein can be observed (FIG. 4D). This depletion becomes even more striking at P6, when regenerative potential is permanently lost from the cochlear sensory epithelia (32).

Conditional Loss of Yap in the Inner Ear Results in Formation of Significantly Smaller Sensory Organs.

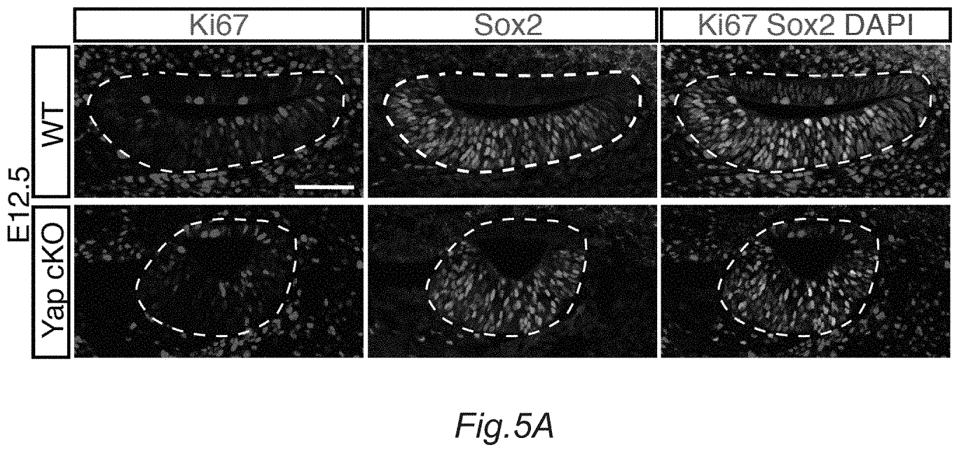

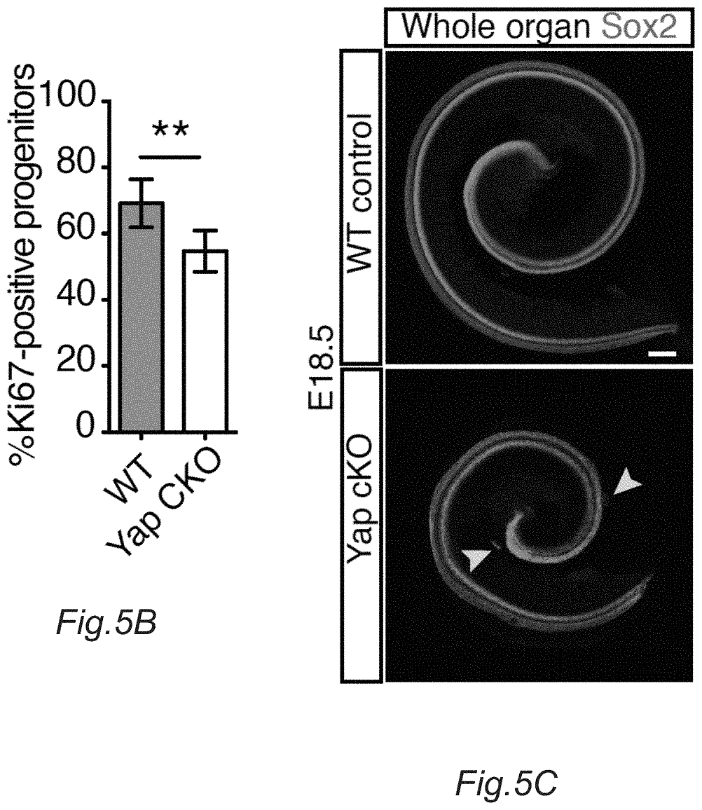

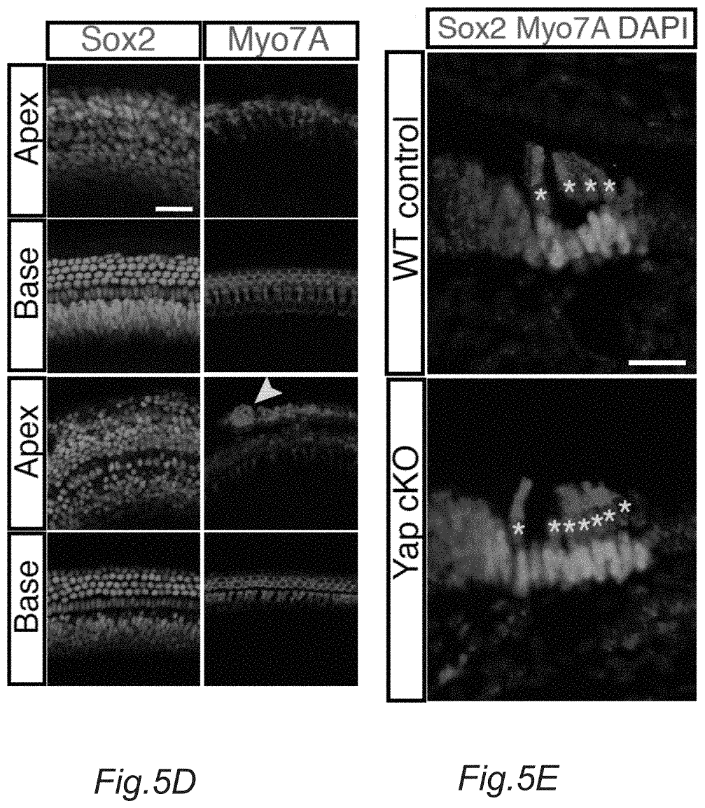

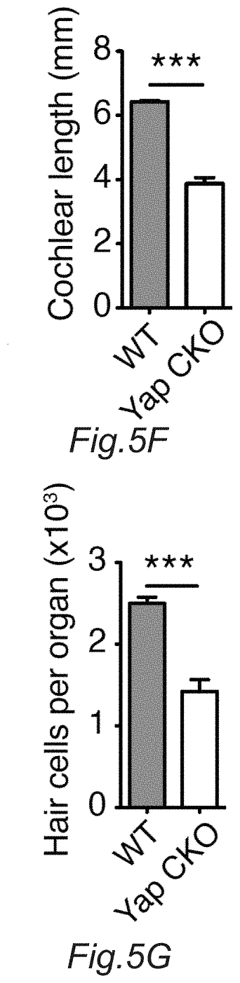

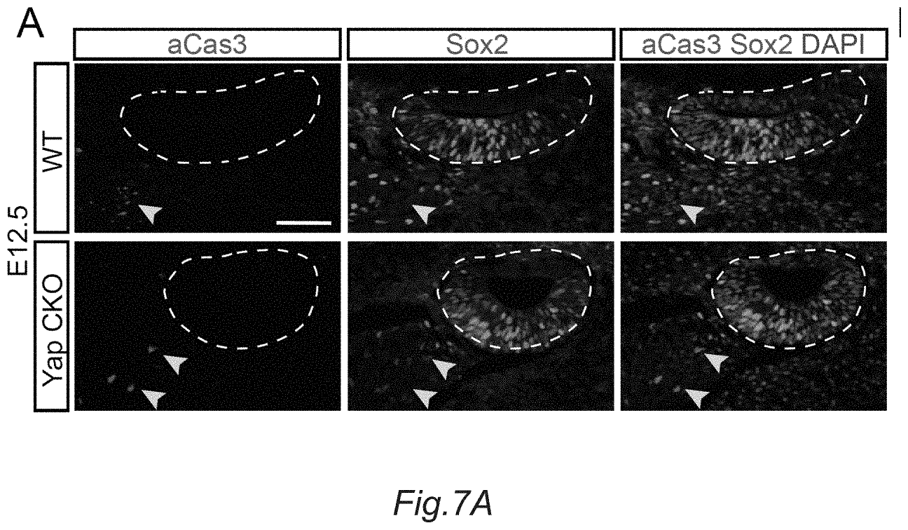

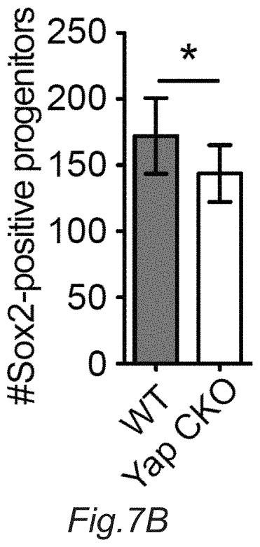

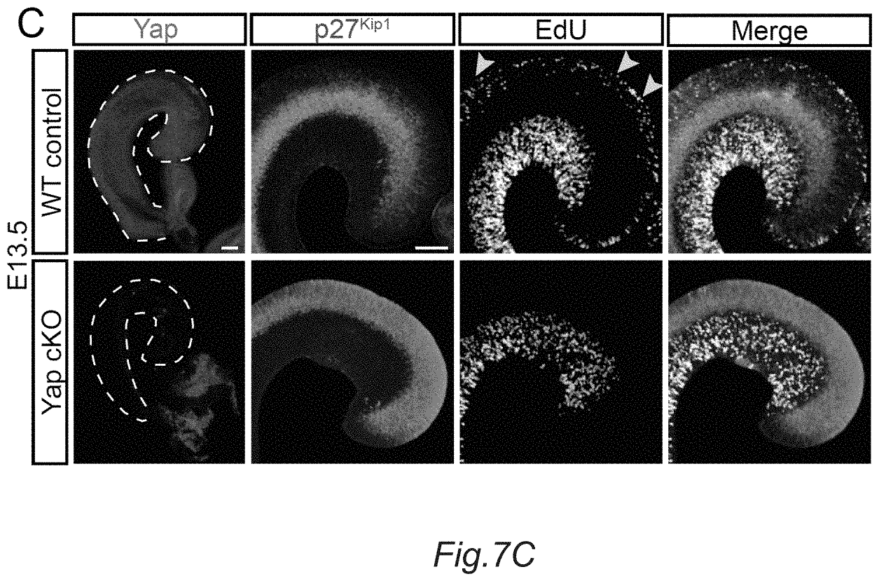

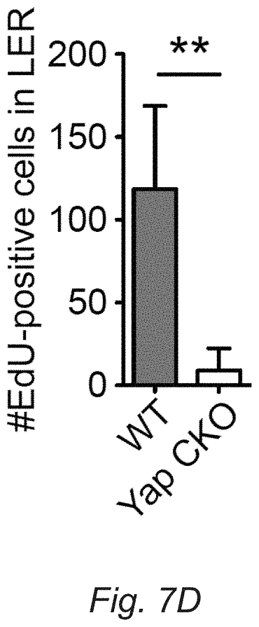

To directly test the role of the Yap/Tead complex in driving progenitor cell proliferation, we generated conditional knockout mice deficient for Yap in the sensory organs of the inner ear using Pax2-Cre and Yap^{fl/fl} mice (33)(34). Consistent with previous reports (7)(8), at E12.5 on average 70% of Sox2-positive sensory progenitor cells in the mid-base of the cochlear duct were actively cycling in Cre-negative, phenotypically wild type littermates (FIG. 5A, B). The percentage of mitotic cells in the Sox2-positive domain decreased over 20% in conditional Yap knockouts (p<0.01; n=9). This decrease in cell proliferation was accompanied by a significant reduction in the total number of Sox2-positive cells (FIGS. 7A, B; p<0.05; n=9). However, we did not observe apoptotic cells within the cochlear duct of either WT or Yap CKO littermates, as shown by the absence of active caspase 3 labeling (FIG. 7A; n=6).

The efficiency of Pax2-Cre-driven recombination was confirmed by demonstrating an absence of the Yap protein in Yap CKO cochleae at E13.5 (FIG. 7C). It is noted that at this stage p27^{Kip1} expression expanded to the abneural domain in the apex of the cochlear duct where no EdU incorporation was observed in the knockouts (FIGS. 7C, D). Nevertheless, upregulation of p27^{Kip1} and cell cycle exit in the prosensory domain still occurred in a wave spreading from apex-to-base in the Yap mutants, suggesting no direct correlation between loss of Yap and transcriptional Cdkn1b upregulation.

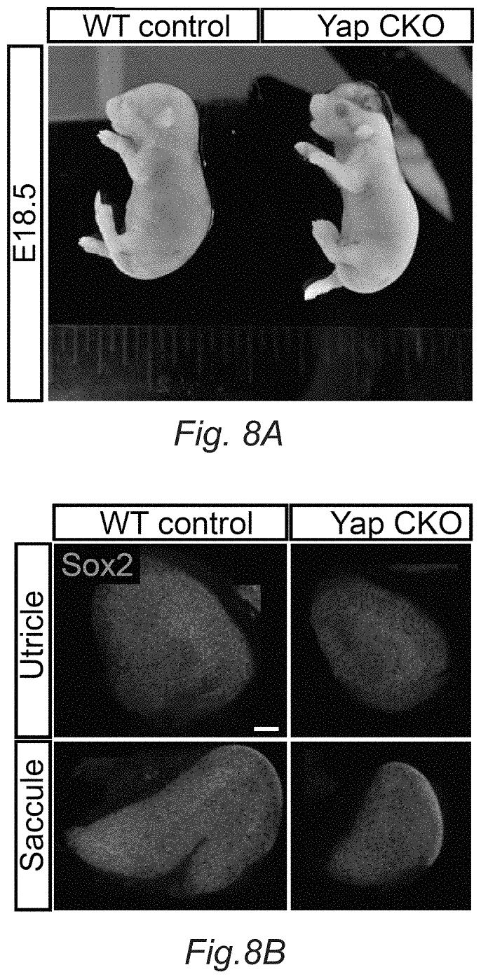

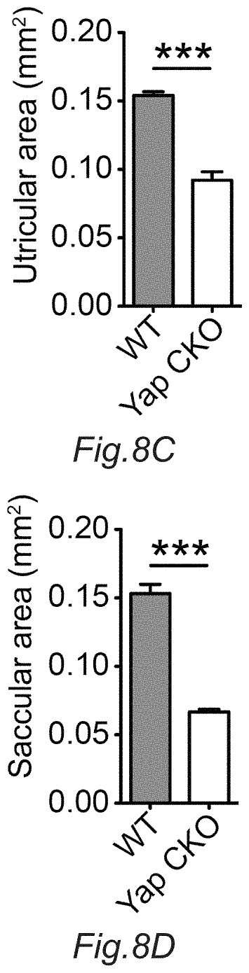

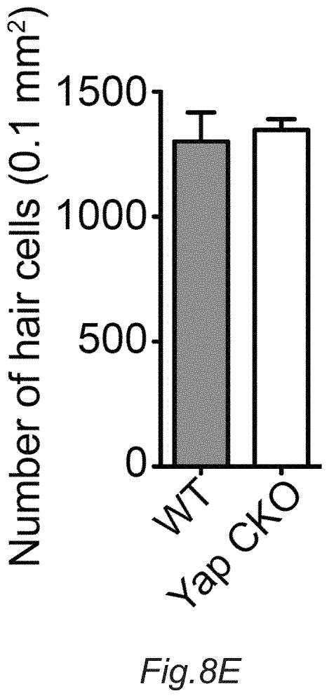

Consistent with the reported pattern of Pax2-Cre expression (33), by later stages of embryonic development Yap CKO animals exhibited midbrain/hindbrain defects and died shortly after birth (FIG. 8A). At E18.5, decreased numbers of the sensory progenitors in conditional Yap mutants manifested in a drastic reduction in the size of the organ of Corti (FIG. 5C, F). However, the pattern of cellular differentiation remained largely intact, with four rows of hair cells and underlying rows of supporting cells detected throughout the entire length of the cochlear duct (FIG. 5C, D). Although the overall number of hair cells was reduced proportionally to the reduction in cochlear length (FIG. 5G), we consistently observed ectopic hair cells and supporting cells on the abneural side of the cochlear duct at the apex, where expanded p27^{Kip1} expression was detected at E13.5 (FIG. 5C-E). Confirming our previous observation that Yap controls growth of the vestibular organs (6), the utricle and saccule were also significantly smaller in Yap CKO mice (FIG. 8B-D). Nevertheless, the hair cell density remained unchanged in these organs (FIG. 8E).

Collectively, these observations strongly suggested that while p27^{Kip1} upregulation serves as the major driver of cell cycle exit in the prosensory domain of the cochlear duct, Yap signaling controls the number of progenitor cells to be formed in the auditory and vestibular sensory organs to regulate their final size.

Constitutive Activation of Yap Via Intraventricular Brain Viral Injection Triggers Cell Cycle Re-Entry in the Postnatal Sensory Epithelia of the Inner Ear.

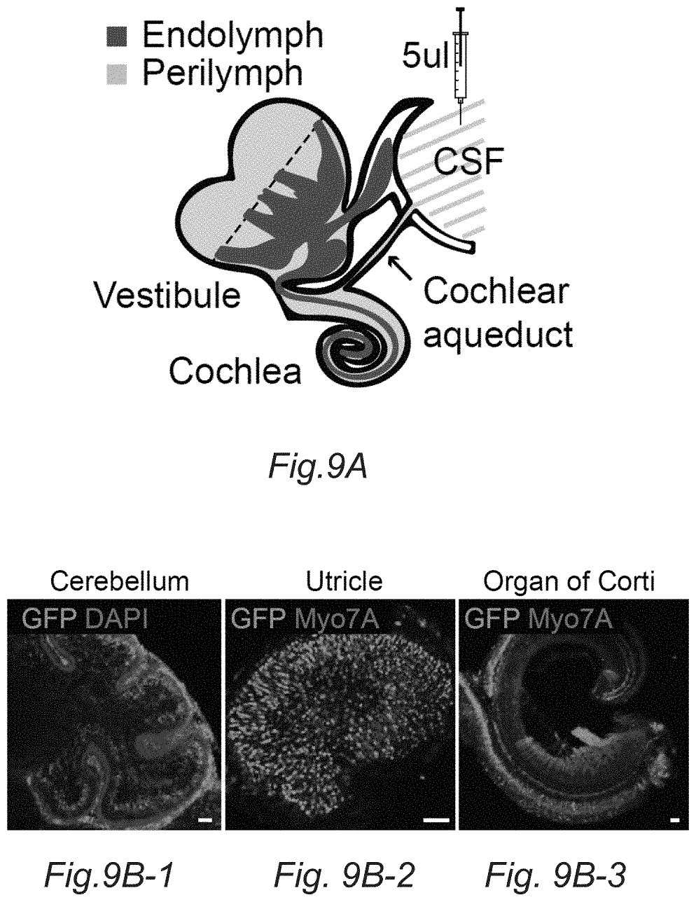

If loss of the Yap/Tead transcription complex causes cell cycle exit in the sensory epithelia of the inner ear, preventing Yap degradation should result in prolonged cell proliferation and cell cycle re-entry in the postmitotic sensory organs. Therefore, to analyze the function of Yap/Tead complex in vivo postnatally, viral vectors were utilized for gene delivery into the inner ear. Round window, posterior semicircular canal, or intraventricular injections are currently used to achieve gene transfer into hair cells and supporting cells. These procedures require an invasive surgery and are labor-intensive, time-consuming, and low-throughput. Because inner ear perilymph is connected directly to the cerebrospinal fluid via the cochlear aqueduct, it was tested whether virus injected intraventricularly will spread into the inner ear in neonatal mice. In brief, 5 μl of the Anc80-GFP virus (37) was injected freehand into the lateral ventricle of p1-p6 neonatal mice anesthetized on ice (38). Using this new method, efficient gene delivery into the central nervous system and both vestibular and auditory sensory epithelia was achieved. The Anc80 vector has been previously described to predominantly infect hair cells, while supporting cells remained uninfected (37). Importantly, we also demonstrated that intraventricular gene delivery in Pou4f3^{DTR/+} mice (39), in which hair cells were killed by diphtheria toxin injection 1 day prior, resulted in effective gene transfer in the residual supporting cells (FIG. 9 C,D).

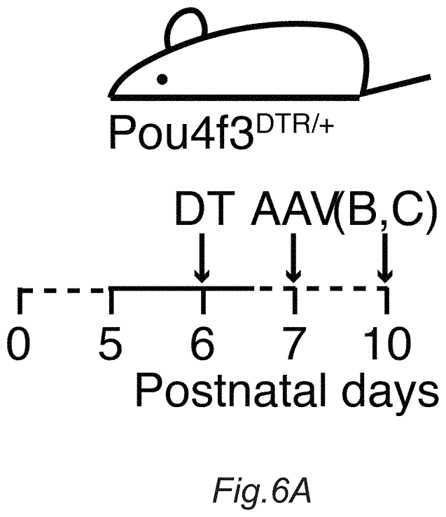

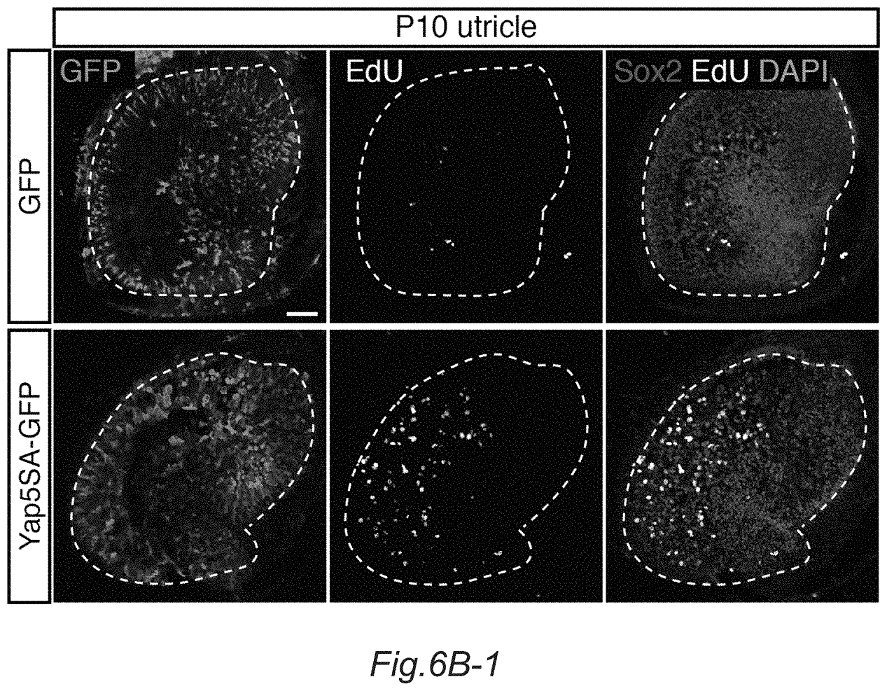

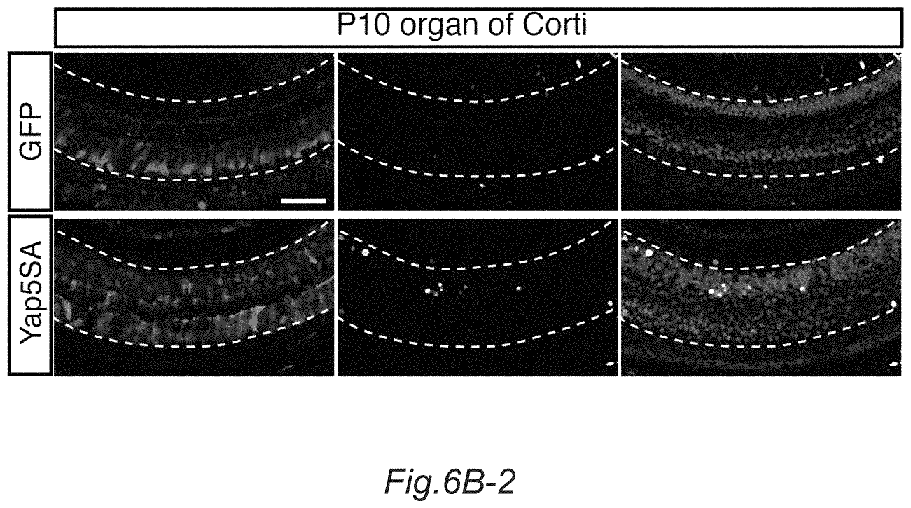

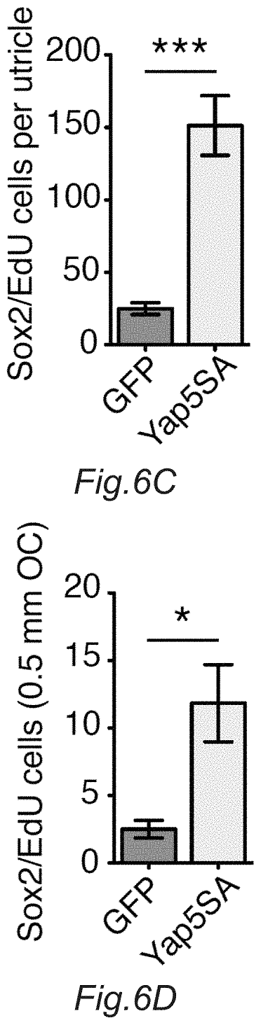

Using this new viral delivery method, the effects of Yap signaling activation in the inner ear sensory epithelia after hair cell ablation was tested (FIG. 6A). Diphtheria toxin was administered at P6, the stage at which spontaneous regeneration was no longer observed in the organ of Corti in vivo (32). The following day, Anc80-GFP control or Anc80-Yap5SA-GFP virus was administered intraventricularly to the animals carrying the DTR allele. The animals were injected with EdU and sacrificed three days after viral injections. It was demonstrated that constitutively activate Yap, Yap5SA, expression, resulted in robust supporting cell cell-cycle re-entry in the utricular macula, where numerous Sox2 and EdU positive supporting cells were observed (FIG. 6B, C). Cell cycle re-entry was also initiated upon Yap5SA overexpression in Sox2-positive cells in Kolliker's organ and in the organ of Corti, albeit at a lower rate (FIG. 6B-1, D). These data demonstrate that activation of Yap signaling is sufficient to drive supporting cell proliferation in postnatal inner ear sensory organs—a process normally blocked in mammals, but necessary for sensory hair cell regeneration in non-mammalian vertebrates.

### DISCUSSION

Aspects of the present invention characterize the role of the Yap/Tead complex in maintaining the proliferative state of organ of Corti progenitors prior to the establishment of the post-mitotic prosensory domain. We demonstrate that Tead transcription factors directly control expression of cell cycle genes and that re-activation of Yap/Tead signaling is sufficient to prevent cell cycle exit during embryogenesis, and to induce supporting cell proliferation postnatally.

Prior to our work, most research was focused on Wnt signaling as a major regulator of progenitor self-renewal in the sensory epithelia of the inner ear (40). Similar to Yap signaling, canonical Wnt activity is detected at high levels in the prosensory domain of the cochlear duct prior to p27^{Kip1} up-regulation, and is reduced thereafter (41). In addition, both genetic and small molecule activation of Wnt signaling is sufficient to promote cell proliferation in the embryonic and neonatal organ of Corti (41)(42)(43)(44). Although we show that loss of Yap results in significant reduction in the proportion of dividing cells within the Sox2-positive prosensory domain of the cochlear duct at E12.5, the progenitor cells do not completely lose their mitotic capacity in the absence of Yap/Tead signaling. It is, therefore, likely that other mitogenic pathways, such as Wnt, act in parallel with Yap/Tead signaling to maintain the self-renewal state in the cochlear prosensory cells. It is important to note, however, that Taz, a closely related homologue of Yap, is also expressed in the sensory progenitors of the cochlea duct (FIG. 4A). Taz can drive cell proliferation in complex with Tead transcription factors (12)(13), thus it may partially compensate for loss of Yap in conditional inner ear knockouts. Interestingly, a recent study demonstrated that conditional inactivation of Ctnnb1 (β-catenin), as early as E10.5, does not result in significant reduction in the organ of Corti length, nor in supporting and hair cell numbers, suggesting normal progenitor cell proliferation in the absence of canonical Wnt signaling (45). In stark contrast, conditional loss of Yap drastically affects the organ of Corti size, suggesting the dominance of Yap/Taz/Tead signaling in driving cell proliferation during development.

Given the similar patterns of Yap degradation and p27^{Kip1} up-regulation in the cochlea, it is attractive to propose a functional relationship between the pathways, or to hypothesize that stability of both proteins is regulated by the same upstream mechanisms. Recent work indicates that YAP can be polyubiquitinated by the SCF-SKP2 E3 ligase complex, which enhances its nuclear translocation and Yap/Tead complex stability (46). The SCF-SKP2 complex is a well-established regulator of the protein levels of cyclins and cyclin-dependent kinase inhibitors, and can degrade p27^{Kip1} (47). Consistent with this observation, our data demonstrate an almost four-fold decrease in Skp2 expression in post-mitotic organ of Corti progenitor cells. Moreover, the Yap/Tead complex was recently shown to directly regulate Skp2 transcription in human breast cancers, where high Yap and low-p21^{Cip1}/p27^{Kip1} levels of expression are correlated (48). Our data support these observations, as we identify the Skp2 gene as one of the direct Tead targets in the sensory epithelia using the Cut&Run assay and show that conditional loss of Yap in the inner ear results in expansion of p27^{Kip1} expression in the apex of the cochlear duct.

Our data does not support the idea that Yap/Tead degradation initiates the apical-to-basal wave of transcriptional p27^{Kip1} activation—a form of cell cycle control unique to the organ of Corti (8). It does, however, suggest a similar transcriptional level of control for the Yap/Tead pathway in the developing organ of Corti. In particular, we show that Yap expression is downregulated, while expression of the core Hippo kinases and adaptor proteins is upregulated as progenitor cells transition into a post-mitotic state. More research is needed to understand the intertwined, yet distinct, roles for Yap and p27^{Kip1} as upstream regulators of cell cycle in the inner ear.

In addition to expanding the mechanistic understanding of the early inner ear sensory epithelia development, our work provides insight into how regenerative responses can be initiated in inner ear sensory tissue. Adult mammalian supporting cells in both vestibular and auditory epithelia lack the capacity to re-enter the cell cycle to regenerate lost hair cells in vivo—the main way by which hearing and vestibular functions are restored in birds (49)(50)(51)(52). Despite considerable effort, there has been only limited success in inducing such proliferative responses in postnatal mammalian sensory organs in vivo, mostly via constitutive activation of Wnt signaling (42)(43)(44). Recent research clearly demonstrates that the Hippo pathway antagonizes Wnt to control tissue growth and regeneration (53)(54)(55)(56). Our previous work (6), as well as our new data on transgenic and viral induction of Yap5SA expression in the inner ear provides clear evidence for Hippo as a major repressor of regeneration in the tissue, and explains why ablation of p27^{Kip1} is not sufficient to substantially relieve the block on supporting cell proliferation (9)(10).

Although constitutive activation of Yap clearly does not represent a therapeutically relevant strategy for augmenting proliferative regeneration in the sensory epithelia, locally administered small molecule inhibition of the Hippo and p27^{Kip1} pathways may represent a viable strategy for mammalian hair cell regeneration.

Materials and Methods

Animal Care and Strains

Experiments were conducted in accordance with the policies of the Institutional Animal Care and Use Committees of the Keck School of Medicine of USC. p27^{Kip1}-GFP mice were previously described in our laboratory (8). Sox2-CreER, and Sox2-GFP mice were obtained from the Jackson laboratory. Pax2-Cre mice (33) were provided by Dr. Groves, Baylor College of Medicine. Yap^{fl/fl} mice were described previously (34).

Immunohistochemistry and EdU Labeling

Embryos were extracted from euthanized mice and placed into ice-cold Hank's balanced salt solution (HBSS, Life Technologies). Inner ears were identified, and cochleae or utricles were dissected as described previously (57). Utricles and cochlear ducts were fixed in 4% formaldehyde for 20 min at RT. Whole inner ears were fixed ON at 4° C., treated with 30% sucrose ON at 4° C., embedded in Tissue-Tek O.C.T. (Sakura), and frozen in liquid-nitrogen vapor. Whole mount sensory epithelia or 10 m frozen sections were then blocked for 1 hr to ON at RT in a blocking solution of the following composition: 5% normal donkey serum (Sigma-Aldrich), 0.5% Triton X 100 (Sigma-Aldrich), and 20 mM Tris-Buffered Saline (10×TBS; Bio-Rad) at pH 7.5. The following primary antibodies-goat anti-Sox2 (Santa Cruz and R&D), mouse anti-p27^{Kip1} (Thermo Fisher Scientific), rabbit anti-Myo7A (Proteus Bioscience), rabbit anti-GFP (Torrey Pines Biolabs), rabbit anti-Ki67 (Abcam), active caspase 3 (R&D Systems), goat anti-Flag (Novus Biologicals), mouse anti-Yap (Santa Cruz), and rabbit anti-Yap (Cell Signaling)—were reconstituted in blocking solution and applied overnight at 4° C. Samples were washed with 20 mM TBS supplemented with 0.1% Tween 20 (Sigma-Aldrich), after which Alexa Fluor-labeled secondary antibodies (Life Technologies) were applied in the same solution for 2 hr at room temperature. Nuclei were stained with 3 m DAPI (Sigma-Aldrich).

EdU pulse-chase experiments were initiated by a single intraperitoneal injection of 50 ng EdU (Abcam) per gram of body mass. Animals were sacrificed at the indicated times and the cells in the sensory epithelia were analyzed by Click-iT EdU labeling (Life Technologies).

Western Blotting

The epithelial preparations of the cochlear ducts were isolated at E12.5 and E14.5 in ice-cold Hank's balanced salt solution (HBSS, Life Technologies) supplemented with a cocktail of protease inhibitors (cOMPLETE mini, EDTA free; Roche). The preparations were then lysed in 50 uL RIPA lysis buffer supplemented with the same cocktail of protease inhibitors (Roche) for 30 min at 4° C. Protein lysates were sonicated thrice at low power for 10 s each with the sample kept on ice between the sonications. The total protein concentration in each sample was determined by the BCA assay (Thermo Fisher). A NuPAGE™ 12% Bis-Tris Protein Gel (Thermo Fisher) was used to resolve the proteins in 5 ug of each sample. The proteins were transferred to a nitrocellulose membrane (BioRad) and blocked for 1 hr at room temperature in a 5% solution of skim-milk powder (Sigma-Aldrich) in TBS supplemented with 0.1% Tween 20 (Sigma-Aldrich) or in Odyssey blocking buffer (Licor). The primary antibodies—rabbit anti-Yap (Cell Signaling), rabbit anti-pYap (Ser127; Cell Signaling), rabbit anti-Lats1 (Cell Signaling), rabbit anti-pLats1 (Ser909; Cell Signaling), rabbit anti-Mst1 (Cell Signaling), and rabbit anti-H3 (Millipore)—were reconstituted at 1:10000 in the blocking buffer and the membranes were incubated over night at 4° C. After five 30 min washes at room temperature in TBS supplemented with 0.1% Tween 20, the anti-rabbit HRP secondary antibody (Millipore) or anti-rabbit IR800 dye (Licor) was applied in TBST for 1 hr at RT. Horseradish-peroxidase activity was detected with the Amersham ECL Western Blotting System (GE Healthcare Life Sciences).

Adenoviral Gene Transfer

The pAnc80L65AAP vector (37)(Addgene plasmid 92307) was used to create adeno-associated viral vectors containing the full-length coding sequence of GFP or Yap5SA-GFP fusion protein (Addgene plasmid 33093) under the control of a cytomegalovirus promoter. Viral particles were packaged in HEK 293T cells and purified by CsCl-gradient centrifugation followed by dialysis (Viral Vector Core Facility, Sanford-Burnham Medical Research Institute). Each animal was injected at P7 into the lateral ventricle with 5 ul of virus at a titer of 10¹² PFU/mL as described previously for infection of CNS neurons (38).

RNA-Sequencing Analysis

Total RNA from FACS-purified organ of Corti progenitor cells was extracted using Quick-RNA MicroPrep kit (Zymo Research) and stored up to 2 weeks at −80° C. RNA samples were then processed for library preparation with QIAseq FX Single Cell RNA Library Kit (Qiagen) and the quality of the library was confirmed using a Bioanalyzer (Quick Biology Inc.). Two biological replicates were collected for each, E12.0 and E13.5, stage, and at least 20 million 150 base-paired-end reads were sequenced for each replicate. Reads were mapped to GRCm38/mm10 genome assembly using STAR (58). Differentially expressed protein coding genes were identified by DESeq2 (FDR<0.05)(59). For data visualization, principal components analysis was performed by PCAExplorer using top 1000 most significantly differentially expressed genes (60).

ATAC-Sequencing and CUT&RUN

The ATAC-seq protocol was described previously (61). Tn5 transposase was expressed and purified according to Picelli et al., 2014 and was used with the following modifications. Briefly, five thousand FACS-purified progenitor cells were used for each of three biological replicates sequenced for E12.0 and E13.5 organ of Corti. Tn5 transposition was performed for 20 min at 37° C. At least 30 million paired-end reads were sequenced for each sample.

The CUT&RUN method for in situ chromatin immunoprecipitation was described previously (19)(20), and was used to profile Tead occupancy and lysine 27 acetylation on histone 3 (H3K27Ac) of the chromatin in E12.0 and E13.5 progenitors. At least 20,000 cells were used for each of two Tead CUT&RUNs, 5,000 cells were used for each of two biological replicates of H3K27Ac for E12.0 and E13.5 progenitor cells, and 1,000 cells were used as IgG only control. Protein A/MNase fusion protein was a kind gift from Dr. Henikoff's laboratory. Rb anti-panTead (Cell Signaling) and rb anti-H3K27Ac (Active Motif) antibodies were used. To construct CUT&RUN libraries, Accel-NGS 2S plus DNA prep kits with single index and MIDs (Swift Bioscience) was used. At least 20 million paired-end reads were sequenced for each sample.

Encode pipelines were adapted for alignment and QC for ATAC-seq and CUT&RUN data. Briefly, the next generation reads were trimmed to 37 bp and aligned to GRCm38/mm10 genome assembly (58). PCR duplicates were removed based on genomic coordinates for ATAC-seq, or by MIDs using UMI-tools for CUT&RUN (63). Peaks were called by Model-based analysis of ChIP-Seq (MACS2) with FDR<0.01 and the dynamic lambda (--nolambda) option for individual replicates (64). IDR or pooled peaks were identified between the biological replicates for each sample and used for the downstream analysis. BigWig files were generated with deepTools (65). Individual genomic loci were visualized in IGV (66) using fold-enrichment tracks generated in MACS2 (64) (67). Heatmaps were generated with deepTools based on normalized bigWig signal files. To identify transcription factor binding enrichments in the subsets of the genomic regions, whole genome was used as a background in HOMER (18).

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. D. K. Wu, M. W. Kelley, Molecular mechanisms of inner ear development. Cold Spring Harb. Perspect. Biol. 4, a008409 (2012).
2. R. J. Ruben, Development of the inner ear of the mouse: a radioautographic study of terminal mitoses. Acta Otolaryngol. (Stockh.), Suppl 220:1-44 (1967).
3. X. Yang, et al., Establishment of planar cell polarity is coupled to regional cell cycle exit and cell differentiation in the mouse utricle. Sci. Rep. 7, 43021 (2017).
4. J. C. Burns, D. On, W. Baker, M. S. Collado, J. T. Corwin, Over half the hair cells in the mouse utricle first appear after birth, with significant numbers originating from early postnatal mitotic production in peripheral and striolar growth zones. J. Assoc. Res. Otolaryngol. JARO 13, 609-627 (2012).
5. K. Gnedeva, A. J. Hudspeth, SoxC transcription factors are essential for the development of the inner ear. Proc. Natl. Acad. Sci. U.S.A 112, 14066-14071 (2015).
6. K. Gnedeva, A. Jacobo, J. D. Salvi, A. A. Petelski, A. J. Hudspeth, Elastic force restricts growth of the murine utricle. eLife 6 (2017).
7. P. Chen, N. Segil, p27(Kip1) links cell proliferation to morphogenesis in the developing organ of Corti. Dev. Camb. Engl. 126, 1581-1590 (1999).
8. Y.-S. Lee, F. Liu, N. Segil, A morphogenetic wave of p27Kip1 transcription directs cell cycle exit during organ of Corti development. Dev. Camb. Engl. 133, 2817-2826 (2006).
9. H. Lowenheim, et al., Gene disruption of p27(Kip1) allows cell proliferation in the postnatal and adult organ of corti. Proc. Natl. Acad. Sci. U.S.A 96, 4084-4088 (1999).
10. H. Laine, M. Sulg, A. Kirjavainen, U. Pirvola, Cell cycle regulation in the inner ear sensory epithelia: role of cyclin D1 and cyclin-dependent kinase inhibitors. Dev. Biol. 337, 134-146 (2010).
11. Z. Meng, T. Moroishi, K.-L. Guan, Mechanisms of Hippo pathway regulation. Genes Dev. 30, 1-17 (2016).
12. B. Zhao, et al., TEAD mediates YAP-dependent gene induction and growth control. Genes Dev. 22, 1962-1971 (2008).
13. L. M. Koontz, et al., The Hippo effector Yorkie controls normal tissue growth by antagonizing scalloped-mediated default repression. Dev. Cell 25, 388-401 (2013).
14. H. Oh, et al., Genome-wide association of Yorkie with chromatin and chromatin-remodeling complexes. Cell Rep. 3, 309-318 (2013).
15. G. G. Galli, et al., YAP Drives Growth by Controlling Transcriptional Pause Release from Dynamic Enhancers. Mol. Cell 60, 328-337 (2015).
16. F. Zanconato, et al., Genome-wide association between YAP/TAZ/TEAD and AP-1 at enhancers drives oncogenic growth. Nat. Cell Biol. 17, 1218-1227 (2015).
17. K. Arnold, et al., Sox2(+) adult stem and progenitor cells are important for tissue regeneration and survival of mice. Cell Stem Cell 9, 317-329 (2011).
18. S. Heinz, et al., Simple combinations of lineage-determining transcription factors prime cis-regulatory elements required for macrophage and B cell identities. Mol. Cell 38, 576-589 (2010).
19. P. J. Skene, S. Henikoff, An efficient targeted nuclease strategy for high-resolution mapping of DNA binding sites. eLife 6 (2017).
20. P. J. Skene, J. G. Henikoff, S. Henikoff, Targeted in situ genome-wide profiling with high efficiency for low cell numbers. Nat. Protoc. 13, 1006-1019 (2018).
21. M. P. Creyghton, et al., Histone H3K27ac separates active from poised enhancers and predicts developmental state. Proc. Natl. Acad. Sci. U.S.A 107, 21931-21936 (2010).
22. A. Rada-Iglesias, et al., A unique chromatin signature uncovers early developmental enhancers in humans. Nature 470, 279-283 (2011).
23. C. Y. McLean, et al., GREAT improves functional interpretation of cis-regulatory regions. Nat. Biotechnol. 28, 495-501 (2010).
24. S. L. Prescott, et al., Enhancer divergence and cis-regulatory evolution in the human and chimp neural crest. Cell 163, 68-83 (2015).
25. M.-C. Lai, W.-C. Chang, S.-Y. Shieh, W.-Y. Tarn, DDX3 regulates cell growth through translational control of cyclin E1. Mol. Cell. Biol. 30, 5444-5453 (2010).
26. H. Fang, et al., RecQL4-Aurora B kinase axis is essential for cellular proliferation, cell cycle progression, and mitotic integrity. Oncogenesis 7, 68 (2018).
27. C. J. Sherr, D-type cyclins. Trends Biochem. Sci. 20, 187-190 (1995).
28. A. W. Hunter, et al., The kinesin-related protein MCAK is a microtubule depolymerase that forms an ATP-hydrolyzing complex at microtubule ends. Mol. Cell 11, 445-457 (2003).
29. A. Subramanian, et al., Gene set enrichment analysis: a knowledge-based approach for interpreting genome-wide expression profiles. Proc. Natl. Acad. Sci. U.S.A 102, 15545-15550 (2005).
30. J. Kwan, et al., DLG5 connects cell polarity and Hippo signaling protein networks by linking PAR-1 with MST1/2. Genes Dev. 30, 2696-2709 (2016).
31. I. M. Moya, G. Halder, Hippo-YAP/TAZ signalling in organ regeneration and regenerative medicine. Nat. Rev. Mol. Cell Biol. 20, 211-226 (2019).
32. B. C. Cox, et al., Spontaneous hair cell regeneration in the neonatal mouse cochlea in vivo. Dev. Camb. Engl. 141, 816-829 (2014).
33. T. Ohyama, A. K. Groves, Generation of Pax2-Cre mice by modification of a Pax2 bacterial artificial chromosome. Genes. N. Y. N 2000 38, 195-199 (2004).
34. N. Zhang, et al., The Merlin/NF2 tumor suppressor functions through the YAP oncoprotein to regulate tissue homeostasis in mammals. Dev. Cell 19, 27-38 (2010).
35. T. O. Monroe, et al., YAP Partially Reprograms Chromatin Accessibility to Directly Induce Adult Cardiogenesis In Vivo. Dev. Cell 48, 765-779.e7 (2019).
36. B. I. Carlborg, J. C. Farmer, Transmission of cerebrospinal fluid pressure via the cochlear aqueduct and endolymphatic sac. Am. J. Otolaryngol. 4, 273-282 (1983).
37. L. D. Landegger, et al., A synthetic AAV vector enables safe and efficient gene transfer to the mammalian inner ear. Nat. Biotechnol. 35, 280-284 (2017).
38. J.-Y. Kim, S. D. Grunke, Y. Levites, T. E. Golde, J. L. Jankowsky, Intracerebroventricular viral injection of the neonatal mouse brain for persistent and widespread neuronal transduction. J. Vis. Exp. JoVE, 51863 (2014).
39. J. S. Golub, et al., Hair cell replacement in adult mouse utricles after targeted ablation of hair cells with diphtheria toxin. J. Neurosci. Off. J. Soc. Neurosci. 32, 15093-15105 (2012).
40. V. Munnamalai, D. M. Fekete, Wnt signaling during cochlear development. Semin. Cell Dev. Biol. 24, 480-489 (2013).
41. B. E. Jacques, et al., A dual function for canonical Wnt/?-catenin signaling in the developing mammalian cochlea. Dev. Camb. Engl. 139, 4395-4404 (2012).
42. R. Chai, et al., Wnt signaling induces proliferation of sensory precursors in the postnatal mouse cochlea. Proc. Natl. Acad. Sci. U.S.A 109, 8167-8172 (2012).
43. F. Shi, L. Hu, A. S. B. Edge, Generation of hair cells in neonatal mice by ?-catenin overexpression in Lgr5-positive cochlear progenitors. Proc. Natl. Acad. Sci. U.S.A 110, 13851-13856 (2013).
44. W. Ni, et al., Extensive Supporting Cell Proliferation and Mitotic Hair Cell Generation by In Vivo Genetic Reprogramming in the Neonatal Mouse Cochlea. J. Neurosci. Off. J. Soc. Neurosci. 36, 8734-8745 (2016).
45. L. Jansson, et al., ?-Catenin is required for radial cell patterning and identity in the developing mouse cochlea. Proc. Natl. Acad. Sci. U.S.A 116, 21054-21060 (2019).
46. F. Yao, et al., SKP2- and OTUD1-regulated non-proteolytic ubiquitination of YAP promotes YAP nuclear localization and activity. Nat. Commun. 9, 2269 (2018).
47. T. Cardozo, M. Pagano, The SCF ubiquitin ligase: insights into a molecular machine. Nat. Rev. Mol. Cell Biol. 5, 739-751 (2004).
48. W. Jang, T. Kim, J. S. Koo, S.-K. Kim, D.-S. Lim, Mechanical cue-induced YAP instructs Skp2-dependent cell cycle exit and oncogenic signaling. EMBO J. 36, 2510-2528 (2017).
49. J. T. Corwin, D. A. Cotanche, Regeneration of sensory hair cells after acoustic trauma. Science 240, 1772-1774 (1988).
50. B. M. Ryals, E. W. Rubel, Hair cell regeneration after acoustic trauma in adult Coturnix quail. Science 240, 1774-1776 (1988).
51. P. Weisleder, E. W. Rubel, Hair cell regeneration after streptomycin toxicity in the avian vestibular epithelium. J. Comp. Neurol. 331, 97-110 (1993).
52. J. S. Stone, E. W. Rubel, Cellular studies of auditory hair cell regeneration in birds. Proc. Natl. Acad. Sci. U.S.A 97, 11714-11721 (2000).
53. X. Varelas, et al., The Hippo pathway regulates Wnt/beta-catenin signaling. Dev. Cell 18, 579-591 (2010).
54. T. Heallen, et al., Hippo pathway inhibits Wnt signaling to restrain cardiomyocyte proliferation and heart size. Science 332, 458-461 (2011).
55. M. Imajo, K. Miyatake, A. Iimura, A. Miyamoto, E. Nishida, A molecular mechanism that links Hippo signalling to the inhibition of Wnt/?-catenin signalling. EMBO J. 31, 1109-1122 (2012).
56. B. R. Kuo, E. M. Baldwin, W. S. Layman, M. M. Taketo, J. Zuo, In Vivo Cochlear Hair Cell Generation and Survival by Coactivation of ?-Catenin and Atoh1. J. Neurosci. Off. J. Soc. Neurosci. 35, 10786-10798 (2015).
57. K. Gnedeva, A. J. Hudspeth, N. Segil, Three-dimensional Organotypic Cultures of Vestibular and Auditory Sensory Organs. J. Vis. Exp. JoVE (2018) https:/doi.org/10.3791/57527.
58. A. Dobin, et al., STAR: ultrafast universal RNA-seq aligner. Bioinforma. Oxf. Engl. 29, 15-21 (2013).
59. M. I. Love, W. Huber, S. Anders, Moderated estimation of fold change and dispersion for RNA-seq data with DESeq2. Genome Biol. 15, 550 (2014).
60. F. Marini, H. Binder, pcaExplorer: an R/Bioconductor package for interacting with RNA-seq principal components. BMC Bioinformatics 20, 331 (2019).
61. J. D. Buenrostro, P. G. Giresi, L. C. Zaba, H. Y. Chang, W. J. Greenleaf, Transposition of native chromatin for fast and sensitive epigenomic profiling of open chromatin, DNA-binding proteins and nucleosome position. Nat. Methods 10, 1213-1218 (2013).
62. S. Picelli, et al., Tn5 transposase and tagmentation procedures for massively scaled sequencing projects. Genome Res. 24, 2033-2040 (2014).
63. T. Smith, A. Heger, I. Sudbery, UMI-tools: modeling sequencing errors in Unique Molecular Identifiers to improve quantification accuracy. Genome Res. 27, 491-499 (2017).
64. J. Feng, T. Liu, B. Qin, Y. Zhang, X. S. Liu, Identifying ChIP-seq enrichment using MACS. Nat. Protoc. 7, 1728-1740 (2012).
65. F. Ramirez, F. Dundar, S. Diehl, B. A. Graning, T. Manke, deepTools: a flexible platform for exploring deep-sequencing data. Nucleic Acids Res. 42, W187-191 (2014).
66. H. Thorvaldsdóttir, J. T. Robinson, J. P. Mesirov, Integrative Genomics Viewer (IGV): high-performance genomics data visualization and exploration. Brief. Bioinform. 14, 178-192 (2013).
67. Y. Zhang, et al., Model-based analysis of ChIP-Seq (MACS). Genome Biol. 9, R137 (2008).

What is claimed is:

1. A method for inducing sensory receptor regeneration, the method comprising:
identifying a subject in need of regeneration of inner ear sensory epithelia; and
activating Yap/Tead signaling in the subject.

2. The method of claim 1 wherein Yap/Tead signaling is activated by introducing an expression vector into the subject such that the expression vector contacts inner ear sensory epithelia in a sufficient amount to induce regeneration thereof, the expression vector encoding a constitutively active YAP gene.

3. The method of claim 2 wherein the expression vector is introduced by injection.

4. The method of claim 2 wherein the expression vector is introduced by round window, posterior semicircular canal, or intraventricular injections.

5. The method of claim 2 wherein the expression vector includes an expression control sequence operably linked to the constitutively active YAP gene.

6. The method of claim 2 wherein the expression vector is a virus selected from the group consisting of adenoviruses, adeno-associated viruses, retroviruses, lentiviruses, vaccinia viruses, vesicular stomatitis virus, herpes viruses, maraba virus, or papilloma viruses.

7. The method of claim 2 wherein the expression vector is adeno-associated viral vector.

8. The method of claim 2 wherein the expression vector is an Anc80 virus.

9. The method of claim 2 wherein the expression vector is a plasmid.

10. The method of claim 2 wherein the constitutively active YAP gene is a nucleotide sequence having SEQ ID NO: 1 or a nucleotide sequence that is substantially similar to SEQ ID NO: 1 while maintaining at least one serine to alanine mutation in SEQ ID NO: 1.

11. The method of claim 10 wherein the nucleotide sequence that is substantially similar to SEQ ID NO: 1 is at least 70% identical to SEQ ID NO: 1 while maintaining at least one serine to alanine mutation in SEQ ID NO: 1.

12. The method of claim 10 wherein the nucleotide sequence that is substantially similar to SEQ ID NO: 1 is at least 95% identical to SEQ ID NO: 1 while maintaining at least one serine to alanine mutation in SEQ ID NO: 1.

13. The method of claim 10 wherein the constitutively active YAP gene is a nucleotide sequence having SEQ ID NO: 2 or a nucleotide sequence that is substantially similar to SEQ ID NO: 2 while maintaining the 127SA mutation from SEQ ID NO: 2.

14. The method of claim 10 wherein the nucleotide sequence that is substantially similar to SEQ ID NO: 2 is at least 70% identical to SEQ ID NO: 2 while maintaining the 127SA mutation from SEQ ID NO: 2.

15. The method of claim 2 wherein the constitutively active YAP gene is a nucleotide sequence encoding polypeptides having SEQ ID NO: 6 or SEQ ID NO: 7.

16. The method of claim 1 wherein the Yap/Tead signaling is activated by inhibiting or activating one or more upstream regulators.

17. The method of claim 1 wherein the Yap/Tead signaling is activated by inhibiting or downregulating expression of one or more of Lats1/2, Mst1/2, Sav1, Mob1a/b, Nf2, Wwc1, Vgll4, or Dchs1.

18. The method of claim 1 wherein expression of one or more of Lats1/2, Mst1/2, Sav1, Mob1a/b, Nf2, Wwc1, Vgll4, or Dchs1 is inhibited or downregulated by targeting gene expression or RNA translation.

19. The method of claim 1 wherein the Yap/Tead signaling is activated by activating or upregulating expression of one or more of Dlg5, Ajuba, Wtip, or Tead1-4 is to activate Yap/Tead signaling.

20. A pharmaceutical composition for activating Yap/Tead signaling in a subject, the pharmaceutical composition comprising:
a pharmaceutically acceptable carrier liquid; and
an expression vector encoding a constitutively active YAP gene, the expression vector being dispersed in the pharmaceutically acceptable carrier liquid at a sufficient concentration to deliver a pharmaceutically effective amount to the subject.
